(19) United States
(12) Patent Application Publication
(10) Pub. No.: US 2010/0087446 A1
(43) Pub. Date: Apr. 8, 2010

(54) 2-SUBSTITUTED INDOLE DERIVATIVES AS CALCIUM CHANNEL BLOCKERS

(76) Inventors: Prasun K. Chakravarty, Edison, NJ (US); Joseph L. Duffy, Cranford, NJ (US); Min K. Park, Whippany, NJ (US); Sriram Tyagarajan, Edison, NJ (US); Bishan Zhou, Hoboken, NJ (US)

Correspondence Address: MERCK AND CO., INC, P O BOX 2000, RAHWAY, NJ, 07065-0907, US

(21) Appl. No.: 12/597,081

(22) PCT Filed: Apr. 22, 2008
(86) PCT No.: PCT/US08/05156

§ 371 Date: Oct. 22, 2009

Related U.S. Application Data
(60) Provisional application No. 60/926,303, filed on Apr. 26, 2007.

(51) Int. Cl. A61K 31/404; C07D 209/04; C07D 401/04; C07D 403/06; A61K 31/4439; A61K 31/496; A61P 25/00; A61P 25/08

(52) U.S. Cl. 514/254.09; 548/510; 546/277.4; 544/373; 514/415; 514/339

(57) ABSTRACT

2-Substituted indole derivatives represented by Formula I, or pharmaceutically acceptable salts thereof. Pharmaceutical compositions comprise an effective amount of the instant compounds, either alone, or in combination with one or more other therapeutically active compounds, and a pharmaceutically acceptable carrier. Methods of treating conditions associated with, or caused by, calcium channel activity, including, for example, acute pain, chronic pain, visceral pain, inflammatory pain, neuropathic pain, urinary incontinence, itchiness, allergic dermatitis, epilepsy, diabetic neuropathy, irritable bowel syndrome, depression, anxiety, multiple sclerosis, bipolar disorder and stroke, comprise administering an effective amount of the present compounds, either alone, or in combination with one or more other therapeutically active compounds.

### CROSS REFERENCE TO RELATED APPLICATIONS

This application claims priority under 35 U.S.C. §119 from U.S. Application No. 60/926,303, filed Apr. 26, 2007.

### FIELD OF THE INVENTION

This invention relates to a series of 2-substituted indole derivatives. In particular, this invention relates to 2-substituted indole derivatives that are N-type voltage-activated calcium channel blockers useful for the treatment of a variety of pain conditions including chronic and neuropathic pain. The compounds of the present invention also display activity in connection with on T-type voltage-activated calcium channels. The compounds described in this invention are also useful for the treatment of conditions including disorders of bladder function, pruritis, itchiness, allergic dermatitis and disorders of the central nervous system (CNS) such as stroke, epilepsy, essential tremor, schizophrenia, Parkinson's disease, manic depression, bipolar disorder, depression, anxiety, sleep disorder, diabetic neuropathy, hypertension, cancer, diabetes, infertility and sexual dysfunction.

### BACKGROUND TO THE INVENTION

Ion channels control a wide range of cellular activities in both excitable and non-excitable cells (Hille, 2002). Ion channels are attractive therapeutic targets due to their involvement in many physiological processes. In excitable cells, the coordinated function of the resident set of ion channels controls the electrical behavior of the cell. Plasma membrane calcium channels are members of a diverse superfamily of voltage gated channel proteins. Calcium channels are membrane-spanning, multi-subunit proteins that allow controlled entry of Ca2+ ions into cells from the extracellular fluid. Excitable cells throughout the animal kingdom, and at least some bacterial, fungal and plant cells, possess one or more types of calcium channel. Nearly all “excitable” cells in animals, such as neurons of the central nervous system (CNS), peripheral nerve cells and muscle cells, including those of skeletal muscles, cardiac muscles, and venous and arterial smooth muscles, have voltage-dependent calcium channels. Voltage-gated calcium channels provide an important link between electrical activity at the plasma membrane and cell activities that are dependent on intracellular calcium, including muscle contraction, neurotransmitter release, hormone secretion and gene expression. Voltage-gated calcium channels serve to integrate and transduce plasma membrane electrical activity into changes in intracellular calcium concentration, and can do this on a rapid time scale.

Multiple types of calcium channels have been identified in mammalian cells from various tissues, including skeletal muscle, cardiac muscle, lung, smooth muscle and brain. A major family of this type is the L-type calcium channels, whose function is inhibited by the familiar classes of calcium channel blockers (dihydropyridines such as nifedipine, phenylalkylamines such as verapamil, and benzothiazepines such as diltiazem). Additional classes of plasma membrane calcium channels are referred to as T, N, P, Q and R. The “T-type” (or “low voltage-activated”) calcium channels are so named because they open for a shorter duration (T=transient) than the longer (L=long-lasting) openings of the L-type calcium channels. The L, N, P and Q-type channels activate at more positive potentials (high voltage activated) and display diverse kinetics and voltage-dependent properties.

Because of the crucial role in cell physiology, modulation of calcium channel activity can have profound effects. Mutations in calcium channel subunits have been implicated in a number of genetic diseases including familial hemiplegic migraine, spinocerebellar ataxia, Timothy Syndrome, incomplete congenital stationary night blindness and familial hypokalemic periodic paralysis. Modulation of voltage-gated calcium channels by signaling pathways, including c-AMP-dependent protein kinases and G proteins is an important component of signaling by hormones and neurotransmitters (Catterall, 2000). Pharmacological modulation of calcium channels can have significant therapeutic effects, including the use of L-type calcium channel (Caᵥ1.2) blockers in the treatment of hypertension (Hockerman, et al., 1997) and more recently, use of Ziconitide, a peptide blocker of N-type calcium channels (Caᵥ2.2), for the treatment of intractable pain (Staats, et al., 2004). Zicontide is derived from Conotoxin, a peptide toxin isolated from cone snail venom, must be applied by intrathecal injection to allow its access to a site of action in the spinal cord and to minimize exposure to channels in the autonomic nervous system that are involved in regulating cardiovascular function. Ziconotide has also been shown to highly effective as a neuroprotective agent in rat models of global and focal ischemia (Colbume et. Al., Stroke (1999) 30, 662-668) suggesting that modulation of N-type calcium channels (Caᵥ2.2) has implication in the treatment of stroke.

Clinical and preclinical experiments with ziconitide and related peptides confirm a key role of N-type calcium channels in transmitting nociceptive signals into the spinal cord. Identification of N-type calcium channel blockers that can be administered systemically, and effectively block N-type calcium channels in the nociceptive signaling pathway, while sparing N-type calcium channel function in the periphery would provide important new tools for treating some forms of pain. The present invention describes blockers of N-type calcium channels (Caᵥ2.2) that display functional selectivity by blocking N-type calcium channel activity needed to maintain pathological nociceptive signaling, while exhibiting a lesser potency at blocking N-type calcium channels involved in maintaining normal cardiovascular function.

There are three subtypes of T-type calcium channels that have been identified from various warm blooded animals including rat [J Biol. Chem.276(6) 3999-4011 (2001); Eur J Neurosci 11(12):4171-8(1999); reviewed in Cell Mol Life Sci 56(7-8):660-9 (1999)]. These subtypes are termed α1G, α1H, and α1I, and the molecular properties of these channels demonstrate 60-70% homology in the amino acid sequences. The electrophysiological characterization of these individual subtypes has revealed differences in their voltage-dependent activation, inactivation, deactivation and steady-state inactivation levels and their selectivity to various ions such as barium (J Biol. Chem.276(6) 3999-4011 (2001)). Pharmacologically, these subtypes have shown differing sensitivities to blockade by ionic nickel. These channel subtypes are also expressed in various forms due to their ability to undergo various splicing events during their assembly (J Biol. Chem.276 (6) 3999-4011 (2001)).

T-type calcium channels have been implicated in pathologies related to various diseases and disorders, including epilepsy, essential tremor, pain, neuropathic pain, schizophrenia, Parkinson's disease, depression, anxiety, sleep disorders, sleep disturbances, psychosis, schizophrenia, cardiac arrhythmia, hypertension, pain, cancer, diabetes, infertility and sexual dysfunction (J Neuroscience, 14, 5485 (1994); Drugs Future 30(6), 573-580 (2005); EMBO J, 24, 315-324 (2005); Drug Discovery Today, 11, 5/6, 245-253 (2006)).

### SUMMARY OF THE INVENTION

The present invention is directed to series of 2-substituted indole derivatives which are N-type calcium channel (Cav2.2) blockers useful for the treatment of acute pain, chronic pain, cancer pain, visceral pain, inflammatory pain, neuropathic pain, post-herpetic neuralgia, diabatic neuropathy, trigeminal neuralgia, migrane, fibromyalgia and stroke. The compounds of the present invention also display activities on T-type voltage-activated calcium channels (Cav 3.1 and Cav 3.2). The compounds described in this invention are also useful for the treatment of other conditions, including disorders of bladder function, pruritis, itchiness, allergic dermatitis and disorders of the central nervous system (CNS) such as stroke, epilepsy, essential tremor, schizophrenia, Parkinson's disease, manic depression, bipolar disorder, depression, anxiety, sleep disorder, diabetic neuropathy, hypertension, cancer, diabetes, infertility and sexual dysfunction. This invention also provides pharmaceutical compositions comprising a compound of the present invention, either alone, or in combination with one or more therapeutically active compounds, and a pharmaceutically acceptable carrier.

This invention further comprises methods for the treatment of acute pain, chronic pain, visceral pain, inflammatory pain, neuropathic pain and disorders of the CNS including, but not limited to, epilepsy, manic depression, depression, anxiety and bipolar disorder comprising administering the compounds and pharmaceutical compositions of the present invention.

### DETAILED DESCRIPTION OF THE INVENTION

The compounds of this invention are represented by Formula I:

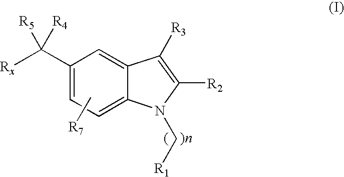

and pharmaceutically acceptable salts, individual enantiomers and diastereomers thereof wherein:
Rₓ is

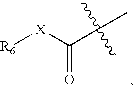

### CN, or CH₂OH;

n is 0-3, where when n=0, R₁ is not H;
X═NR₆, O or is a bond;
R₁ is selected from:
a) hydrogen, C₁-C₆-alkyl or C₃-C₇-cycloalkyl, both optionally substituted with 1 to 3 groups of a substituent selected from C₁-C₄-perfluoroalkyl, C₁-C₆-alkyl, F, Cl, Br, NH₂, NHR₈, NR₈R₉, OH, OR₈, CONHR₈, COOR₈, COR₈, SR₈, SO₂R₁₀, SO₂NHR₈, C₆-C₁₀ aryl or C₅-C₁₀ heteroaryl,
b) C₆-C₁₀ aryl or C₅-C₁₀ heterocycle, both optionally substituted with 1 to 3 groups of a substituent selected from C₁-C₄-perfluoroalkyl, C₁-C₆-alkyl, C₃-C₇-cycloalkyl, F, Cl, Br, NH₂, NHR₈, NR₈R₉, OH, OR₈, CONHR₈, CONR₈R₉, COOR₈, and COR₈,
c) CONR₈R₉, COOR₈ or COR₈, and
d) SOR₁₀, SO₂R₁₀, or SO₂NHR₈;
R₂ is selected from:
(a) C₁-C₆-alkyl, C₃-C₇-cycloalkyl, C₆-C₁₀ aryl or (CH₂)ₙC₅-C₁₀ heterocycle, said alkyl, cycloalkyl, aryl and heteroaryl optionally substituted with 1 to 3 groups of a substituent selected from (O)₀₋₁C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, and COR₈;
(b) CONR₈R₉, COOR₈ or COR₈
R₃ is selected from:
(a) H, C₁-C₄-perfluoroalkyl, C₁-C₆-alkyl and C₃-C₇-cycloalkyl, said alkyl and cycloalkyl optionally substituted with 1 to 3 groups of a substituent selected from C₁-C₄-perfluoroalkyl, C₁-C₆-alkyl, F, Cl, Br, NH₂, NHR₈, NR₈R₉, OR₈, CONHR₈, COOR₈, COR₈, SR₈, SO₂R₁₀, SO₂NHR₈, C₆-C₁₀ aryl and C₅-C₁₀ heteroaryl, NHC(O)(CH₂)ₙOR₈;
(b) CN, CONHR₈, CONR₈R₉, COOR₈ or COR₈;
(c) SOR₁₀, SO₂R₁₀, SR₈, or SO₂ NR₈R₉;
(d) C₆-C₁₀ aryl or (CH₂)ₙC₅-C₁₀ heterocyclyl, both optionally substituted with 1 to 3 groups of C₁-C₄-perfluoroalkyl, C₁-C₆-alkyl, C₆-C₁₀ aryl, F, Cl, Br, CN, NH₂, NHR₈, NR₈R₉OH, OR₈, SO₂R₆, CONHR₈, CONR₈R₉, COOR₈, or COR₈;
R₄ and R₅ are each independently selected from H and C₁-C₆-alkyl, said alkyl optionally substituted with 1 to 3 groups of a substituent selected from C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, and COR₈, or R₄ and R₅ join to form a 3-7 member carbocyclic or heterocyclic ring;
R₆ is selected from H, C₁-C₆-alkyl, C₃-C₇-cycloalkyl, C₁-C₄-alkylaryl, and (CH₂)ₙC₅-C₁₀ heterocyclyl, said alkyl, cycloalkyl, alkylaryl, aryl and heteroaryl optionally substituted with 1 to 3 groups of a substituent selected from C₁-C₄-perfluoroalkyl, CN, F, Cl, Br, NH₂, C₆-C₁₀ aryl, NHR₇, NR₈R₉, OH, OR₈, CONHR₈, CONR₈R₉, COOR₈ and COR₈;
R₇ is selected from H, C₁-C₄-alkyl, C₃-C₇-cycloalkyl, C₁-C₄-perfluoroalkyl, F, Cl, Br, I, NR₈R₉, OR₈, CONHR₈, CONR₈R₉, COOR₈, and COR₈;
R₈ and R₉ are each independently selected from H, C₁-C₆-alkyl, C₃-C₇-cycloalkyl, N(R₆)₂, SO₂R₆, —COOR₆, —C(O)C(R₆)₂OCO₂R₆, C(O)C(C₃₋₇ cycloalkyl)OR₆, C(O)C(C₃₋₇ cycloalkyl)OCO₂R₆, (CH₂)ₙC₆-C₁₀ aryl and (CH₂)ₙC₅-C₁₀ heterocycle, said alkyl, cycloalkyl, aryl and heteroaryl optionally substituted with 1 to 3 groups selected from (O)₀₋₁C₁-C₄-perfluoroalkyl, C₁-C₆-alkyl, F, Cl, Br, CN, NH₂, NHR₈, NR₈R₉, OH, OR₈, (CH₂)ₙC₆-C₁₀ aryl, CONHR₈, CONR₈R₉, COOR₈, or COR₈; and
R₁₀ is selected from C₁-C₄-alkyl, C₃-C₇-cycloalkyl, C₆-C₁₀ aryl and C₅-C₁₀ heteroaryl, said alkyl, cycloalkyl, aryl and heteroaryl optionally substituted with 1 to 3 groups selected from (O)₀₋₁C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, or COR₈.

One aspect of the invention is realized when R₄ and R₅ are both alkyl and all other variables are as originally described.

Another aspect of the invention is realized when Rₓ is

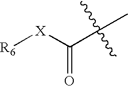

and all other variables are as originally described. A sub-embodiment of this invention is realized when X is NR₆. Another sub-embodiment of this invention is realized when X is —O—. Still another sub-embodiment of this invention is realized when X is a bond.

Another embodiment of this invention is realized when R₆ is hydrogen, C₁-C₆-alkyl, C₃-C₆-cycloalkyl, or (CH₂)ₙC₅-C₁₀ heterocyclyl. A sub-embodiment of this invention is realized when R₆ is hydrogen, C₁-C₆-alkyl, or C₃-C₆-cycloalkyl.

Another aspect of the invention is realized when Rₓ is CN and all other variables are as originally described.

Still another aspect of the invention is realized when Rₓ is CH₂OR₈ and all other variables are as originally described.

Yet another aspect of the invention is realized when n is 0 or 1 and all other variables are as originally described.

Still another aspect of the invention is realized when R₁ is C(O)OR₈, C(O)R₈, C₁-C₆-alkyl, C(O)N(R₈)₂, C₅₋₁₀ heterocycle, or —SO₂R₁₀, and all other variables are as or described, said alkyl and heterocycle optionally substituted. A sub-embodiment of this invention is realized when R₁ is C(O)OR₈. Another sub-embodiment of this invention is realized when R₁ is C(O)R₈. Still another sub-embodiment of this invention is realized when R₁ is C₁-C₆-alkyl, optionally substituted. Yet another sub-embodiment of this invention is realized when R₁ is C(O)N(R₈)₂. Still another embodiment of this invention is realized when R₁ is C₅₋₁₀ heterocycle, optionally substituted.

Another aspect of this invention is realized when R₂ is C₁-C₆-alkyl, and all other variables are as originally described.

Another aspect of the invention is realized when R₂ is C₆-C₁₀ aryl, and all other variables are as originally described.

Still another aspect of the invention is realized when R₂ is (CH₂)ₙC₅-C₁₀ heterocycle, and all other variables are as originally described.

Another aspect of this invention is realized when R₃ is H, C₁-C₆-alkyl, CN, CONR₈R₉, SO₂R₁₀, —COOR₈, —COR₈, or (CH₂)ₙC₅-C₁₀ heterocycle, and all other variables are as originally described. A sub-embodiment of this invention is realized when R₃ is H, or C₁-C₆-alkyl.

Yet another aspect of this invention is realized with the compound of structural formula II:

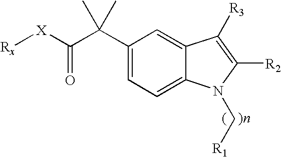

wherein
R₂ is selected from:
C₁-C₆-alkyl, C₆-C₁₀ aryl or (CH₂)ₙC₅-C₁₀ heterocycle, said alkyl, cycloalkyl, aryl and heteroaryl optionally substituted with 1 to 3 groups of a substituent selected from (O)₀₋₁C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, and COR₈; and X, Rₓ, R₁, R₂ and R₃ are as originally described. A sub-embodiment of this invention is realized when R₂ is C₆-C₁₀ aryl; R₁ is C₁-C₆-alkyl, C(O)N(R₈)₂, C₅₋₁₀ heterocycle, COOR₈ or COR_{8,} R₃ is H, C₁-C₆-alkyl, and R₆ is hydrogen, C₁-C₆-alkyl, C₃-C₆-cycloalkyl, or (CH₂)ₙC₅-C₁₀ heterocyclyl. A sub-embodiment of this invention is realized when R₂ is phenyl. Another sub-embodiment of this invention is realized when n is 0 or 1.

As used herein, “alkyl” as well as other groups having the prefix “alk” such as, for example, alkoxy, alkanoyl, alkenyl, and alkynyl means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl,” “alkynyl” and other like terms include carbon chains containing at least one unsaturated C—C bond.

The term “cycloalkyl” refers to a saturated hydrocarbon containing one ring having a specified number of carbon atoms. Examples of cycloalkyl include cyclopropyl, cyclobutyl, cyclopentyl, and cyclohexyl.

The term “C₀₋₄alkyl” includes alkyls containing 4, 3, 2, 1, or no carbon atoms. An alkyl with no carbon atoms is a hydrogen atom substituent when the alkyl is a terminal group and is a direct bond when the alkyl is a bridging group.

The term “alkoxy” as used herein, alone or in combination, includes an alkyl group connected to the oxy connecting atom. The term “alkoxy” also includes alkyl ether groups, where the term ‘alkyl’ is defined above, and ‘ether’ means two alkyl groups with an oxygen atom between them. Examples of suitable alkoxy groups include methoxy, ethoxy, n-propoxy, i-propoxy, n-butoxy, s-butoxy, t-butoxy, methoxymethane (also referred to as ‘dimethyl ether’), and methoxyethane (also referred to as ‘ethyl methyl ether’).

As used herein, “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl.

The term heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

Preferably, heterocycle is selected from 2-azepinonyl, benzimidazolyl, 2-diazapinonyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

“Halogen” refers to fluorine, chlorine, bromine and iodine.

The term “mammal” “mammalian” or “mammals” includes humans, as well as animals, such as dogs, cats, horses, pigs and cattle.

Compounds described herein may contain one or more double bonds and may thus give rise to cis/trans isomers as well as other conformational isomers. The present invention includes all such possible isomers as well as mixtures of such isomers unless specifically stated otherwise.

The compounds of the present invention contain one or more asymmetric centers and may thus occur as racemates, racemic mixtures, single enantiomers, diastereomeric mixtures, and individual diastereomers.

It will be understood that, as used herein, references to the compounds of structural formula I are meant to also include the pharmaceutically acceptable salts, and also salts that are not pharmaceutically acceptable when they are used as precursors to the free compounds or in other synthetic manipulations.

The compounds of the present invention may be administered in the form of a pharmaceutically acceptable salt. The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The pharmaceutical compositions of the present invention comprise compounds of the invention (or pharmaceutically acceptable salts thereof) as an active ingredient, a pharmaceutically acceptable carrier, and optionally one or more additional therapeutic agents or adjuvants. Such additional therapeutic agents can include, for example, i) opiate agonists or antagonists, ii) calcium channel antagonists, iii) 5HT receptor agonists or antagonists, iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) selective serotonin reuptake inhibitors (“SSRI”) and/or selective serotonin and norepinephrine reuptake inhibitors (“SSNRI”), x) tricyclic antidepressant drugs, xi) norepinephrine modulators, xii) lithium, xiii) valproate, xiv) neurontin (gabapentin), and xv) sodium channel blockers. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present compounds and compositions are useful for the treatment of chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, and pain associated with cancer, chemotherapy, HIV and HIV treatment-induced neuropathy. Compounds of this invention may also be utilized as local anesthetics. Compounds of this invention are useful for the treatment of irritable bowel syndrome and related disorders, as well as Crohn's disease.

The instant compounds have clinical uses for the treatment of epilepsy and partial and generalized tonic seizures. They are also useful for neuroprotection under ischaemic conditions caused by stroke or neural trauma and for treating multiple sclerosis. The present compounds are useful for the treatment of tachy-arrhythmias. Additionally, the instant compounds are useful for the treatment of neuropsychiatric disorders, including mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders.

In addition to primates, such as humans, a variety of other mammals can be treated according to the method of the present invention. For instance, mammals including, but not limited to, cows, sheep, goats, horses, dogs, cats guinea pigs, or other bovine, ovine, equine, canine, feline, rodent such as mouse, species can be treated. However, the method can also be practiced in other species, such as avian species (e.g., chickens).

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, neurokinin-1 receptor antagonists, corticotropin releasing factor (CRF) antagonists, and pharmaceutically acceptable salts thereof.

Further, it is understood that compounds of this invention can be administered at prophylactically effective dosage levels to prevent the above-recited conditions and disorders, as well as to prevent other conditions and disorders associated with sodium channel activity.

Creams, ointments, jellies, solutions, or suspensions containing the instant compounds can be employed for topical use. Mouth washes and gargles are included within the scope of topical use for the purposes of this invention.

Dosage levels from about 0.01 mg/kg to about 140 mg/kg of body weight per day are useful in the treatment of inflammatory and neuropathic pain, or alternatively about 0.5 mg to about 7 g per patient per day. For example, inflammatory pain may be effectively treated by the administration of from about 0.01 mg to about 75 mg of the compound per kilogram of body weight per day, or alternatively about 0.5 mg to about 3.5 g per patient per day. Neuropathic pain may be effectively treated by the administration of from about 0.01 mg to about 125 mg of the compound per kilogram of body weight per day, or alternatively about 0.5 mg to about 5.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may ary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors. Such patient-related factors include the age, body weight, general health, sex, and diet of the patient. Other factors include the time and route of administration, rate of excretion, drug combination, and the severity of the particular disease undergoing therapy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I, Ia, Ib, Id or Ie. The compounds of the invention, or pharmaceutically acceptable salts thereof, can also be included in pharmaceutical compositions in combination with one or more therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. As described previously, in preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like may be used; or in the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which solid pharmaceutical carriers are employed. If desired, tablets may be coated by standard aqueous or nonaqueous techniques. In addition to the common dosage forms set out above, controlled release means and/or delivery devices may also be used in administering the instant compounds and compositions.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, and disintegrating agents can be used to form oral solid preparations such as powders, capsules and tablets. Because of their ease of administration, tablets and capsules are advantageous oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet advantageously contains from about 0.1 mg to about 500 mg of the active ingredient and each cachet or capsule advantageously containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and thus should be preserved against the contaminating action of microorganisms such as bacteria and fungi. The carrier can be a solvent or dispersion medium containing, for example, water, ethanol, polyol (e.g. glycerol, propylene glycol and liquid) polyethylene glycol), vegetable oils, and suitable mixtures thereof.

Pharmaceutical compositions of the present invention can be in a form suitable for topical use such as, for example, an aerosol, cream, ointment, lotion, and dusting powder. Further, the compositions can be in a form suitable for use in transdermal devices. These formulations may be prepared, utilizing a compound represented of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid, such as, for example, where the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, and preservatives (including anti-oxidants). Furthermore, other adjuvants can be included to render the formulation isotonic with the blood of the intended recipient. Compositions containing a compound of the invention, or pharmaceutically acceptable salts thereof, can also be prepared in powder or liquid concentrate form.

The compounds and pharmaceutical compositions of this invention have been found to block sodium channels. Accordingly, an aspect of the invention is the treatment and prevention in mammals of conditions that are amenable to amelioration through blockage of neuronal sodium channels by administering an effective amount of a compound of this invention. Such conditions include, for example, acute pain, chronic pain, visceral pain, inflammatory pain and neuropathic pain. The instant compounds and compositions are useful for treating and preventing the above-recited conditions, including acute pain, chronic pain, visceral pain, inflammatory pain and neuropathic pain, in humans and non-human mammals such as dogs and cats. It is understood that the treatment of mammals other than humans refers to the treatment of clinical conditions in non-human mammals that correlate to the above-recited conditions.

Further, as described above, the instant compounds can be utilized in combination with one or more therapeutically active compounds. In particular, the inventive compounds can be advantageously used in combination with i) opiate agonists or antagonists, ii) calcium channel antagonists, iii) 5HT receptor agonists or antagonists, including 5-HT_{1A} agonists or antagonists, and 5-HT_{1A} partial agonists, iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, xiv) norepinephrine reuptake inhibitors, xv) monoamine oxidase inhibitors (MAOIs), xvi) reversible inhibitors of monoamine oxidase (RIMAs), xvii)□-adrenoreceptor antagonists, xviii) atypical anti-depressants, xix) benzodiazepines, xx) corticotropin releasing factor (CRF) antagonists, and xxi) neurontin (gabapentin).

The abbreviations used herein have the following meanings (abbreviations not shown here have their meanings as commonly used unless specifically stated otherwise): Ac (acetyl), Bn (benzyl), Boc (tertiary-butoxy carbonyl), CAMP (cyclic adenosine-3′,5′-monophosphate), DAST ((diethylamino)sulfur trifluoride), DBU (1,8-diazabicyclo[5.4.0]undec-7-ene), DIBAL (diisobutylaluminum hydride), DMAP (4-(dimethylamino)pyridine), DMF (N,N-dimethylformamide), EDC (1-(3-dimethylaminopropyl)-3-ethylcarbodiimide hydrochloride), Et₃N (triethylamine), GST (glutathione transferase), HOBt (1-hydroxybenzotriazole), LAH (lithium aluminum hydride), Ms (methanesulfonyl; mesyl; or SO₂Me), MsO (methanesulfonate or mesylate), NBS (N-bromosuccinimide), NCS (N-chlorosuccinimide), NSAID (non-steroidal anti-inflammatory drug), PDE (Phosphodiesterase), Ph (Phenyl), r.t. or RT (room temperature), Rac (Racemic), SAM (aminosulfonyl; sulfonamide or SO₂NH₂), SPA (scintillation proximity assay), Th (2- or 3-thienyl), TFA (trifluoroacetic acid), THF (Tetrahydrofuran), Thi (Thiophenediyl), TLC (thin layer chromatography), TMEDA (N,N,N′,N′-tetramethylethylenediamine), TMSI (trimethylsilyl iodide), Tr or trityl (N-triphenylmethyl), C₃H₅ (Allyl), Me (methyl), Et (ethyl), n-Pr (normal propyl), i-Pr (isoprOpyl), n-Bu (normal butyl), i-Butyl (isobutyl), s-Bu (secondary butyl), t-Bu (tertiary butyl), c-Pr (cyclopropyl), c-Bu (cyclobutyl), c-Pen (cyclopentyl), c-Hex (cyclohexyl).

The present compounds can be prepared according to the general Schemes provided below as well as the procedures provided in the Examples. The following Schemes and Examples further describe, but do not limit, the scope of the invention.

Unless specifically stated otherwise, the experimental procedures were performed under the following conditions: All operations were carried out at room or ambient temperature; that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) or by high-pressure liquid chromatography-mass spectrometry (HPLC-MS), and reaction times are given for illustration only. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. Broad; etc. In addition, “Ar” signifies an aromatic signal. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### Assay Example 1

### Fluorescent Assay for Cav2.2 Channels Using Potassium Depolarization to Initiate Channel Opening

Human Cav2.2 channels were stably expressed in KEK293 cells along with alpha2-delta and beta subunits of voltage-gated calcium channels. An inwardly rectifying potassium channel (Kir2.3) was also expressed in these cells to allow more precise control of the cell membrane potential by extracellular potassium concentration. At low bath potassium concentration, the membrane potential is relatively negative, and is depolarized as the bath potassium concentration is raised. In this way, the bath potassium concentration can be used to regulate the voltage-dependent conformations of the channels. Compounds are incubated with cells in the presence of low (4 mM) potassium or elevated (12, 25 or 30 mM) potassium to determine the affinity for compound block of resting (closed) channels at 4 mM potassium or affinity for block of open and inactivated channels at 12, 25 or 30 mM potassium. After the incubation period, Cav2.2 channel opening is triggered by addition of higher concentration of potassium (70 mM final concentration) to further depolarize the cell. The degree of state-dependent block can be estimated from the inhibitory potency of compounds after incubation in different potassium concentrations.

Calcium influx through Cav2.2 channels is determined using a calcium-sensitive fluorescent dye in combination with a fluorescent plate reader. Fluorescent changes were measured with either a VIPR (Aurora Instruments) or FLIPR (Molecular Devices) plate reader.

### Protocol

1. Seed cells in Poly-D-Lysine Coated 96- or 384-well plate and keep in a 37° C.-10% CO₂ incubator overnight
2. Remove media¹, wash cells with 0.2 ml (96-well plate) or 0.05 ml (384-well plate) Dulbecco's Phosphate Buffered Saline (D-PBS) with calcium & magnesium (Invitrogen; 14040)
3. Add 0.1 ml (96-well plate) or 0.05 ml (384-well plate) of 4 μM fluo-4 (Molecular Probes; F-14202) and 0.02% Pluronic acid (Molecular Probes; P-3000) prepared in D-PBS with calcium & magnesium (Invitrogen; 14040) supplemented with 10 mM Glucose & 10 mM Hepes/NaOH; pH 7.4
4. Incubate in the dark at 25° C. for 60-70 min
5. Remove dye², wash cells with 0.1 ml (96-well plate) or 0.06 ml (384-well plate) of 4, 12, 25, or 30 mM Potassium Pre-polarization Buffer. (PPB)
6. Add 0.1 ml (96-well plate) or 0.03 ml (384-well plate) of 4, 12, 25, 30 mM PPB. with or without test compound
7. Incubate in the dark at 25° C. for 30 min
8. Read cell plate on VIPR instrument, Excitation=480 nm, Emission=535 nm
9. With VIPR continuously reading, add 0.1 ml (96-well plate) or 0.03 ml (384-well plate) of Depolarization Buffer, which is 2× the final assay concentration, to the cell plate.

Assay Reagents:

| 4 mM K Pre- | | 12 mM K Pre- | | 25 mM K Pre- | | 30 mM K Pre- | | 140 mM K | |
|---|---|---|---|---|---|---|---|---|---|
| Polarization | | Polarization | | Polarization | | Polarization | | Depolarization | |
| Buffer | | Buffer | | Buffer | | Buffer | | Buffer | |
| 146 | mM NaCl | 138 | mM NaCl | 125 | mM NaCl | 120 | mM NaCl | 10 | mM NaCl |
| 4 | mM KCl | 12 | mM KCl | 25 | mM KCl | 30 | mM KCl | 140 | mM KCl |
| 0.8 | mM CaCl₂ | 0.8 | mM CaCl₂ | 0.8 | mM CaCl₂ | 0.8 | mM CaCl₂ | 0.8 | mM CaCl₂ |
| 1.7 | mM MgCl₂ | 1.7 | mM MgCl₂ | 1.7 | mM MgCl₂ | 1.7 | mM MgCl₂ | 1.7 | mM MgCl₂ |
| 10 | mM HEPES | 10 | mM HEPES | 10 | mM HEPES | 10 | mM HEPES | 10 | mM HEPES |

| | | | | |
|---|---|---|---|---|
| pH = 7.2 | pH = 7.2 | pH = 7.2 | pH = 7.2 | pH = 7.2 |

### Assay Example 2

### Electrophysiological Measurement of Block of Cav2.2 Channels Using Automated Electrophysiology Instruments

Block of N-type calcium channels is evaluated utilizing the IonWorks HT 384 well automated patch clamp electrophysiology device. This instrument allows synchronous recording from 384 wells (48 at a time). A single whole cell recording is made in each well. Whole cell recording is established by perfusion of the internal compartment with amphotericin B.

The voltage protocol is designed to detect use-dependent block. A 2 Hz train of depolarizations (twenty 25 ms steps to +20 mV). The experimental sequence consists of a control train (pre-compound), incubation of cells with compound for 5 minutes, followed by a second train (post-compound). Use dependent block by compounds is estimated by comparing fractional block of the first pulse in the train to block of the 20th pulse.

### Protocol

Parallel patch clamp electrophysiology is performed using IonWorks HT (Molecular Devices Corp.) essentially as described by Kiss and colleagues [Kiss et al. 2003; Assay and Drug Development Technologies, 1:127-135]. Briefly, a stable HEK 293 cell line (referred to as CBK) expressing the N-type calcium channel subunits (alphaᵢg, alpha₂-delta, beta_{3a,}) and an inwardly rectifying potassium channel (Kᵢᵣ2.3) is used to record barium current through the N-type calcium channel. Cells are grown in T75 culture plates to 60-90% confluence before use. Cells are rinsed 3× with 10 ml PBS (Ca/Mg-free) followed by addition of 1.0 ml 1× trypsin to the flask. Cells are incubated at 37° C. until rounded and free from plate (usually 1-3 min). Cells are then transferred to a 15 ml conical tube with 13 ml of CBK media containing serum and antibiotics and spun at setting 2 on a table top centrifuge for 2 min. The supernatant is poured off and the pellet of cells is resuspended in external solution (in mM): 120 NaCl, 20 BaCl₂, 4.5 KCl, 0.5 MgCl₂, 10 HEPES, 10 Glucose, pH=7.4). The concentration of cells in suspension is adjusted to achieve 1000-3000 cells per well. Cells are used immediately once they have been resuspended. The internal solution is (in mM): 100 K-Gluconate, 40 KCl, 3.2 MgCl₂, 3 EGTA, 5 HEPES, pH 7.3 with KOH. Perforated patch whole cell recording is achieved by added the perforating agent amphotericin B to the internal solution. A 36 mg/ml stock of amphtericn B is made fresh in DMSO for each run. 166 □l of this stock is added to 50 ml of internal solution yielding a final working solution of 120 ug/ml.

Voltage protocols and the recording of membrane currents are performed using the IonWorks HT software/hardware system. Currents are sampled at 1.25 kHz and leakage subtraction is performed using a 10 mV step from the holding potential and assuming a linear leak conductance. No correction for liquid junction potentials is employed. Cells are voltage clamped at −70 mV for 10 s followed by a 20 pulse train of 25 ms steps to +20 mV at 2 Hz. After a control train, the cells are incubated with compound for 5 minutes and a second train is applied. Use dependent block by compounds is estimated by comparing fractional block of the first pulse to block of the 20th pulse. Wells with seal resistances less than 70 MOhms or less than 0.1 nA of Ba current at the test potential (+20 mV) are excluded from analysis. Current amplitudes are calculated with the IonWorks software. Relative current, percent inhibition and IC50s are calculated with a custom Excel/Sigmaplot macro.

Compounds are added to cells with a fluidics head from a 96-well compound plate. To compensate for the dilution of compound during addition, the compound plate concentration is 3× higher than the final concentration on the patch plate.

Two types of experiments are generally performed: screens and titrations. In the screening mode, 10-20 compounds are evaluated at a single concentration (usually 3 uM). The percent inhibition is calculated from the ratio of the current amplitude in the presence and absence of compound, normalized to the ratio in vehicle control wells. For generation of IC50s, a 10-point titration is performed on 2-4 compounds per patch plate. The range of concentrations tested is generally 0.001 to 20 uM. IC50s are calculated from the fits of the Hill equation to the data. The form of the Hill equation used is: Relative Current=Max−Min)/(1+(conc/IC50)̂slope))+Min. Vehicle controls (DMSO) and 0.3 mM CdCl₂ (which inhibits the channel completely) are run on each plate for normalization purposes and to define the Max and Min.

### Assay Example 3

### Electrophysiological Measurement of Block of Cav2.2 Channels Using Whole Cell Voltage Clamp and Using PatchXpress Automated Electrophysiology Instrument

Block of N-type calcium channels is evaluated utilizing manual and automated (PatchXpress) patch clamp electrophysiology. Voltage protocols are designed to detect state-dependent block. Pulses (50 ms) are applied at a slow frequency (0.067 Hz) from polarized (−90 mV) or depolarized (−40 mV) holding potentials. Compounds which preferentially block inactivated/open channels over resting channels will have higher potency at −40 mV compared to −90 mV.

### Protocol:

A stable HEK 293 cell line (referred to as CBK) expressing the N-type calcium channel subunits (alpha_{2B}, alpha₂-delta, beta_{3a,}) and an inwardly rectifying potassium channel (Kᵢᵣ2.3) is used to record barium current through the N-type calcium channel. Cells are grown either on poly-D-lysine coated coverglass (manual EP) or in T75 culture plates (PatchXpress). For the PatchXpress, cells are released from the flask using tryspin. In both cases, the external solution is (in mM): 120 NaCl, 20 BaCl₂, 4.5 KCl, 0.5 MgCl₂, 10 HEPES, 10 Glucose, pH 7.4 with NaOH. The internal solution is (in mM): 130 CsCl, 10 EGTA, 10 HEPES, 2 MgCl₂, 3 MgATP, pH 7.3 with CsOH.

Barium currents are measured by manual whole-cell patch clamp using standard techniques (Hamill et. al. Pfluegers Archiv 391:85-100 (1981)). Microelectrodes are fabricated from borosilicate glass and fire-polished. Electrode resistances are generally 2 to 4 MOhm when filled with the standard internal saline. The reference electrode is a silver-silver chloride pellet. Voltages are not corrected for the liquid junction potential between the internal and external solutions and leak is subtracted using the P/n procedure. Solutions are applied to cells by bath perfusion via gravity. The experimental chamber volume is ˜0.2 ml and the perfusion rate is 0.5-2 ml/min. Flow of solution through the chamber is maintained at all times. Measurement of current amplitudes is performed with PULSEFIT software (HEKA Elektronik).

PatchXpress (Molecular Devices) is a 16-well whole-cell automated patch clamp device that operates asynchronously with fully integrated fluidics. High resistance (gigaohm) seals are achieved with 50-80% success. Capacitance and series resistance compensation is automated. No correction for liquid junction potentials is employed. Leak is subtracted using the P/n procedure. Compounds are added to cells with a pipettor from a 96-well compound plate. Voltage protocols and the recording of membrane currents are performed using the PatchXpress software/hardware system. Current amplitudes are calculated with DataXpress software.

In both manual and automated patch clamp, cells are voltage clamped at −40 mV or −90 mV and 50 ms pulses to +20 mV are applied every 15 sec (0.067 Hz). Compounds are added in escalating doses to measure % Inhibition. Percent inhibition is calculated from the ratio of the current amplitude in the presence and absence of compound. When multiple doses are achieved per cell, IC50s are calculated. The range of concentrations tested is generally 0.1 to 30 uM. IC50s are calculated from the fits of the Hill equation to the data. The form of the Hill equation used is: Relative Current=1/(1+(conc/IC50)̂slope)).

### Assay Example 4

### Assay for Cav3.1 and Cav3.2 Channels

The T-type calcium channel blocking activity of the compounds of this invention may be readily determined using the methodology well known in the art described by Xia, et al., Assay and Drug Development Tech., 1(5), 637-645 (2003).

In a typical experiment ion channel function from HEK 293 cells expressing the T-type channel alpha-1G, H, or I (CaV 3.1, 3.2, 3.3) is recorded to determine the activity of compounds in blocking the calcium current mediated by the T-type channel alpha-1G, H, or I (CaV 3.1, 3.2, 3.3). In this T-type calcium (Ca²⁺) antagonist voltage-clamp assay calcium currents are elicited from the resting state of the human alpha-1G, H, or I (CaV 3.1, 3.2, 3.3) calcium channel as follows. Sequence information for T-type (Low-voltage activated) calcium channels are fully disclosed in e.g., U.S. Pat. No. 5,618,720, U.S. Pat. No. 5,686,241, U.S. Pat. No. 5,710,250,U.S. Pat. No. 5,726,035, U.S. Pat. No. 5,792,846, U.S. Pat. No. 5,846,757, U.S. Pat. No. 5,851,824, U.S. Pat. No. 5,874,236, U.S. Pat. No. 5,876,958, U.S. Pat. No. 6,013,474, U.S. Pat. No. 6,057,114, U.S. Pat. No. 6,096,514, WO 99/28342, and J. Neuroscience, 19(6):1912-1921 (1999). Cells expressing the t-type channels were grown in H3D5 growth media which comprised DMEM, 6% bovine calf serum (HYCLONE), 30 micromolar Verapamil, 200 microgram/ml Hygromycin B, 1× Penicillin/Streptomycin. Glass pipettes are pulled to a tip diameter of 1-2 micrometer on a pipette puller. The pipettes are filled with the intracellular solution and a chloridized silver wire is inserted along its length, which is then connected to the headstage of the voltage-clamp amplifier. Trypsinization buffer was 0.05% Trypsin, 0.53 mM EDTA. The extracellular recording solution consists of (mM): 130 mM NaCl, 4 mM KCl, 1mM MgCl2, 2mM CaCl2, 10 mM HEPES, 30 Glucose, pH 7.4. The internal solution consists of (mM): 135 mM CsMeSO4, 1 MgCl2, 10 CsCl, 5 EGTA, 10 HEPES, pH 7.4, or 135 mM CsCl, 2 MgCl2, 3 MgATP, 2 Na2ATP, 1 Na2GTP, 5 EGTA, 10 HEPES, pH 7.4. Upon insertion of the pipette tip into the bath, the series resistance is noted (acceptable range is between 1-4 megaohm). The junction potential between the pipette and bath solutions is zeroed on the amplifier. The cell is then patched, the patch broken, and, after compensation for series resistance (>=80%), the voltage protocol is applied while recording the whole cell Ca2+ current response. Voltage protocols: (1) −80 mV holding potential every 20 seconds pulse to −20 mV for 40 msec duration; the effectiveness of the drug in inhibiting the current mediated by the channel is measured directly from measuring the reduction in peak current amplitude initiated by the voltage shift from −80 mV to −20 mV; (2). −100 mV holding potential every 15 seconds pulse to −20 mV for 40 msec duration; the effectiveness of the drug in inhibiting the current mediated by the channel is measured directly from measuring the reduction in peak current amplitude initiated by the shift in potential from −100 mV to −30 mV. The difference in block at the two holding potentials was used to determine the effect of drug at differing levels of inactivation induced by the level of resting state potential of the cells. After obtaining control baseline calcium currents, extracellular solutions containing increasing concentrations of a test compound are washed on. Once steady state inhibition at a given compound concentration is reached, a higher concentration of compound is applied. % inhibition of the peak inward control Ca2+ current during the depolarizing step to −20 mV is plotted as a function of compound concentration.

The intrinsic T-type calcium channel antagonist activity of a compound which may be used in the present invention may be determined by these assays.

In particular, the compounds of the following examples had activity in antagonizing the T-type calcium channel in the aforementioned assays, generally with an IC₅₀ of less than about 10 uM. Preferred compounds within the present invention had activity in antagonizing the T-type calcium channel in the aforementioned assays with an IC₅₀ of less than about 1 uM. Such a result is indicative of the intrinsic activity of the compounds in use as antagonists of T-type calcium channel activity.

In Vivo Assay: (Rodent CFA model):

Male Sprague Dawley rats (300-400 gm) were administered 200 microl CFA (Complete Freund's Adjuvant) three days prior to the study. CFA is mycobacterium tuberculosis suspended in saline (1:1; Sigma) to form an emulsion that contains 0.5 mg mycobacterium/ml. The CFA was injected into the plantar area of the left hind paw.

Rats are fasted the night before the study only for oral administration of compounds. On the morning of test day using a Ugo Basile apparatus, 2 baseline samples are taken 1 hour apart. The rat is wrapped in a towel. Its paw is placed over a ball bearing and under the pressure device. A foot pedal is depressed to apply constant linear pressure. Pressure is stopped when the rat withdraws its paw, vocalizes, or struggles. The right paw is then tested. Rats are then dosed with compound and tested at predetermined time points. Compounds were prepared in DMSO (15%)/PEG300(60%)/Water(25%) and were dosed in a volume of 2 ml/kg.

Percent maximal possible effect (% MPE) was calculated as: (post-treatment−pre-treatment)/(pre-injury threshold−pre-treatment)×100. The % responder is the number of rats that have a MPE.30% at any time following compound administration. The effect of treatment was determined by one-way ANOVA Repeated Measures Friedman Test with a Dunn's post test.

### Methods of Synthesis:

Compounds of the present invention can be prepared according to the Schemes provided below as well as the procedures provided in the Examples. The substituents are the same as in the above Formulas except where defined otherwise or otherwise apparent to the ordinary skilled artisan.

The novel compounds of the present invention can be readily synthesized using techniques known to those skilled in the art, such as those described, for example, in Advanced Organic Chemistry, March, 5^{th} Ed., John Wiley and Sons, New York, N.Y., 2001; Advanced Organic Chemistry, Carey and Sundberg, Vol. A and B, 3^{rd} Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the present compounds may be prepared using standard synthetic transformations of chemical precursors that are readily available from commercial sources, including Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S.C.); Arcos, (Pittsburgh, Pa.) and Trans World Chemicals (Rockville, Md.).

The procedures described herein for synthesizing the compounds may include one or more steps of protecting group manipulations and of purification, such as, re-crystallization, distillation, column chromatography, flash chromatography, thin-layer chromatography (TLC), radial chromatography and high-pressure chromatography (HPLC). The products can be characterized using various techniques well known in the chemical arts, including proton and carbon-13 nuclear magnetic resonance (¹H and ¹³C NMR), infrared and ultraviolet spectroscopy (IR and UV), X-ray crystallography, elemental analysis and HPLC and mass spectrometry (HPLC-MS). Methods of protecting group manipulation, purification, structure identification and quantification are well known to one skilled in the art of chemical synthesis.

Appropriate solvents are those which will at least partially dissolve one or all of the reactants and will not adversely interact with either the reactants or the product. Suitable solvents are aromatic hydrocarbons (e.g., toluene, xylenes), halogenated solvents (e.g., methylene chloride, chloroform, carbontetrachloride, chlorobenzenes), ethers (e.g., diethyl ether, diisopropylether, tert-butyl methyl ether, diglyme, tetrahydrofuran, dioxane, anisole), nitriles (e.g., acetonitrile, propionitrile), ketones (e.g., 2-butanone, dithyl ketone, tert-butyl methyl ketone), alcohols (e.g., methanol, ethanol, n-propanol, iso-propanol, n-butanol, t-butanol), N,N-dimethyl formamide (DMF), dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, sodium carbonate, cesium carbonate, sodium hydrogen carbonate, and cesium hydrogen carbonate; alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnaesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO.

It is understood that the functional groups present in compounds described in the Schemes below can be further manipulated, when appropriate, using the standard functional group transformation techniques available to those skilled in the art, to provide desired compounds described in this invention.

It is also understood that compounds listed in the Schemes and Tables below that contain one or more stereocenters may be prepared as single enantiomers or diastereomers, or as mixtures containing two or more enantiomers or diastereomers in any proportion.

Other variations or modifications, which will be obvious to those skilled in the art, are within the scope and teachings of this invention. This invention is not to be limited except as set forth in the following claims.

2-substituted indoles described in this invention can be synthesized using a variety of synthetic methods described by Humphrey and Kuethe in Chem. Rev., 2006, 106, 2875-2911. 2-aryl indoles, a sub-class of the 2-substituted indoles of this invention, can be synthesized using Fisher Indole reaction as outlined in Scheme 1.

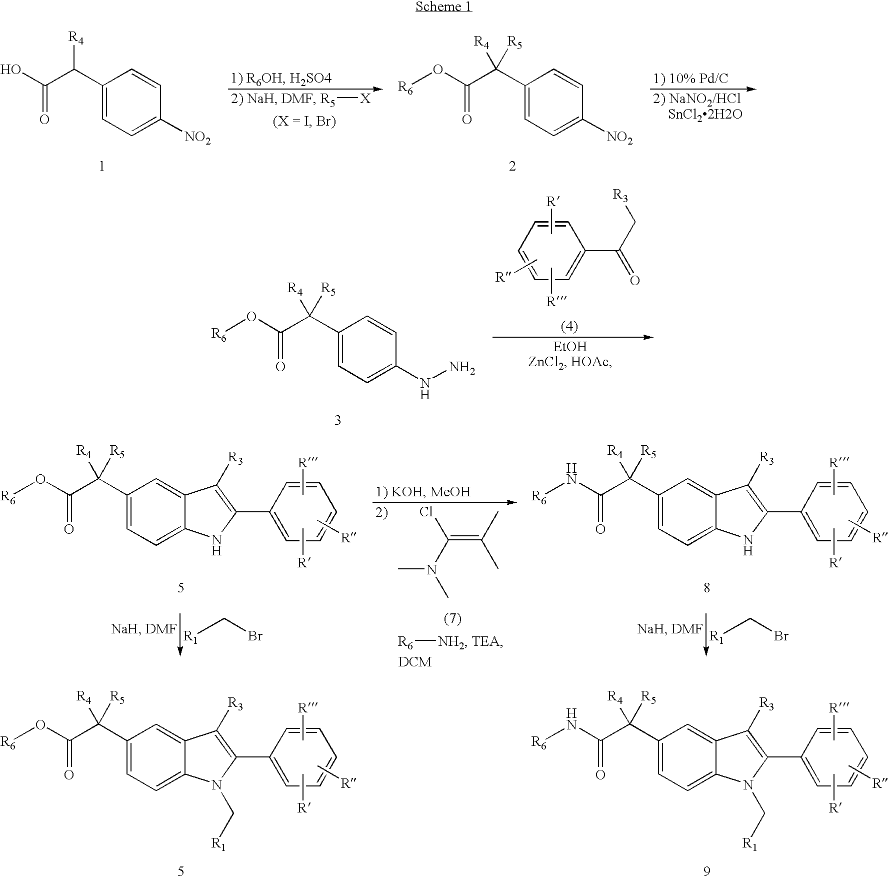

The 4-nitrophenyl propionic acid derivative 1 can be prepared from a variety of commercially available starting materials using the methods described in the following publications [a) Lawrence, N. J., et. al. J. Org. Chem, 2002, 67, 457-464; b) Bowman et. al. Org. Prep. Proced. Int 1990, 22, 636-638; c) Bizzaro, et. al. WO200185707; d) Baron et. al. Tetra. Lett. 2002, 43, 723-726; e) selvakumar et. al. Tetra. Lett. 2001, 42, 8395-8398; f) Davis et. al. J. Org. Chem. 2000, 65, 8704-8708; g) Deshmukh et. al. Org. Prep. Proced. Int 1998, 30, 453-455; h) Bushell et. al. Tetrahedron 1998, 54, 2269-2274]. The 4-nitrophenyl propionic acid derivative 1, thus prepared, can be reacted with an appropriate R₆—OH in the presence of an acid catalyst at temperature ranging from 0° C. to the reflux temperature of the reaction solvent to provide the corresponding ester derivative, which can be then reacted with an alkylating agent R₅—X [e.g., alkyl halides, alkyl sulfonates, benzyl halides, or heteroaryl-alkyl halides] in the presence of an appropriate base (e.g., NaH, Et₃N, diisopropylethylamine, DBU, Na₂CO₃, K₂CO₃ or Cs₂CO₃) in an appropriate solvent (e.g., toluene, THF, dioxane, DMF or DMSO) to provide the product 2.

The nitro group in 2 can be reduced to provide the corresponding aniline 10 (see Scheme 2), which then can be converted to the corresponding aryl hydrazine 3 via a reduction of the diazonium intermediate as outlined in Scheme 1. Subsequent reaction of 3 with an appropriate carbonyl partner 4 in the presence of an acid catalyst under Fisher Indole reaction condition can provide the 2-arylindole 5, which can be alkylated with an appropriate alkylating agent R₁CH₂—X, as outlined, to provide 6. Fisher synthesis of indole 5 can also be prepared in good yields under microwave heating. Hydrolysis of the arylindole 5 can also provide the corresponding carboxylic acid which can be readily converted to amide derivatives 8 by reacting it with an appropriate amine in the presence of an amide forming reagent 7. A variety of other amide forming methods or reagents that are known to one skilled in the art of the synthesis of peptide bonds can also be used. The indole 8 then can be alkylated with an appropriate alkylating agent to provide 9.

The indoles 5 and 8 described above can also be assembled using the conditions of Larock indole synthesis as outlined in Scheme 2. The aniline 11, obtained from 4-nitrophenyl propionic acid derivative 10, can be treated with iodine monochloride to provide the iodoaniline 12 which upon treatment with an appropriate silyl acetylene derivative 13 under Larock condition can lead to the corresponding indole 14. Treatment of 14 with iodine mono-chloride can provide the 2-iodo indole 15 which can be reacted with an appropriate aryl boronate 16 under Pd catalyzed condition to provide the desired indole 5. Similarly, the indole 8 can also be synthesized from the aniline 11 using Larock condition as described by Walsh et. al., in Tetrahedron 2001, 57, 5233-5241. Alternatively, the indole 5 can be subjected to ester hydrolysis conditions to provide the corresponding carboxylic acid 16 which then can be reacted with an appropriate amine in the presence of a suitable amide forming reagent to provide the amide compound 8.

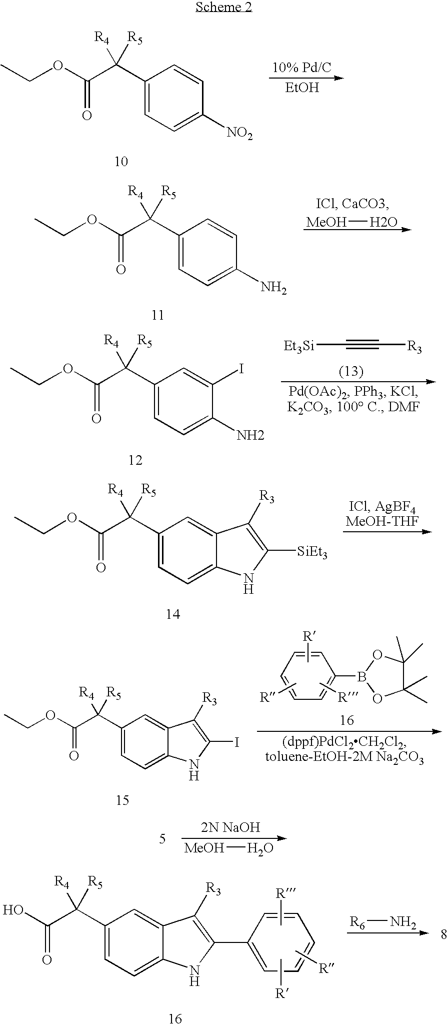

The 2-substituted indole 20 can be prepared from 12 using an alternative metal catalyzed cyclization reaction of an appropriate aryl acetylene intermediate 19 as outlined in Scheme 3.

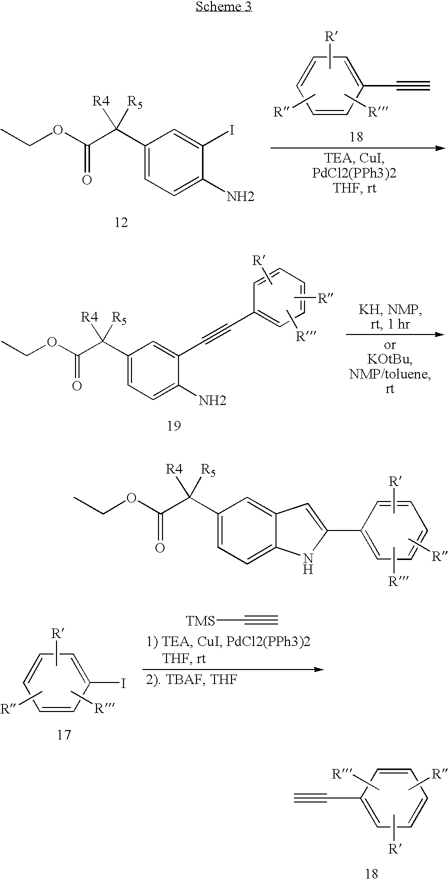

The intermediate 19 can be easily synthesized from the 2-iodo aniline derivative 17 under copper catalyzed reaction of an aryl acetylene intermediate18. The acetylene intermediate 18 can be prepared from iodobenzene and TMS-acetylene using the conditions of Sonogashira reaction (Tetrahedron 2003, 59, 1571).

The indoles 5 and 8 can be reacted with appropriate acylating agents such as, acyl chlorides, acyl imidazoles, acyl carbonates, chloroformates and isocyanates in the presence of an appropriate base (e.g., pyridine, DMAP, trialkyl amines, K₂CO₃, Cs₂CO₃ etc.) to provide the corresponding N¹-acyl indoles 21 and 22 respectively (Scheme 3). Similarly, the N¹-sulfonyl derivatives 23 and 24 can be prepared by reaction of the indoles 5 and 8 respectively, with an appropriate sulfonylating agent in the presence of an appropriate base as outlined in Scheme 4.

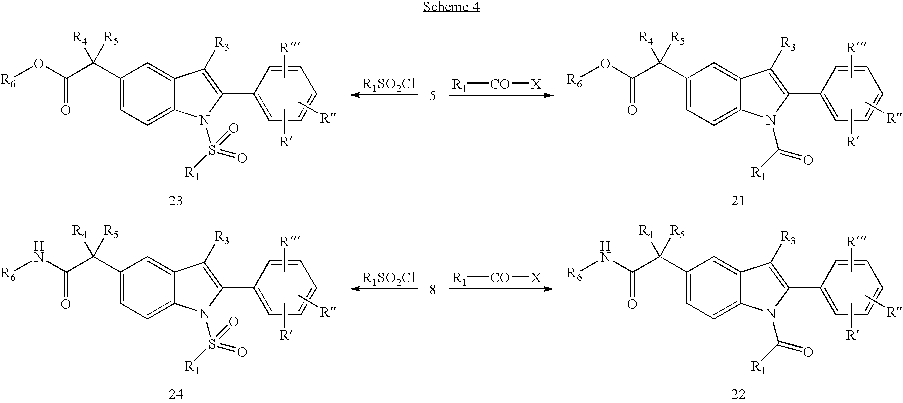

The indoles 26-29 can be prepared from 3 using the reactions outlined in Scheme 5. Reaction of 3 with an appropriate alpha-ketoester 25 under Fisher Indole reaction condition provides the corresponding indole 26, which upon N-alkylation can provide the appropriate indole 27. The ester group in indole 26 can be hydrolyzed, as outlined, and the resulting carboxylic acid compound can be easily converted into appropriate amide derivatives 28. The indole 27 also can be converted into corresponding amide 29 in a similar manner.

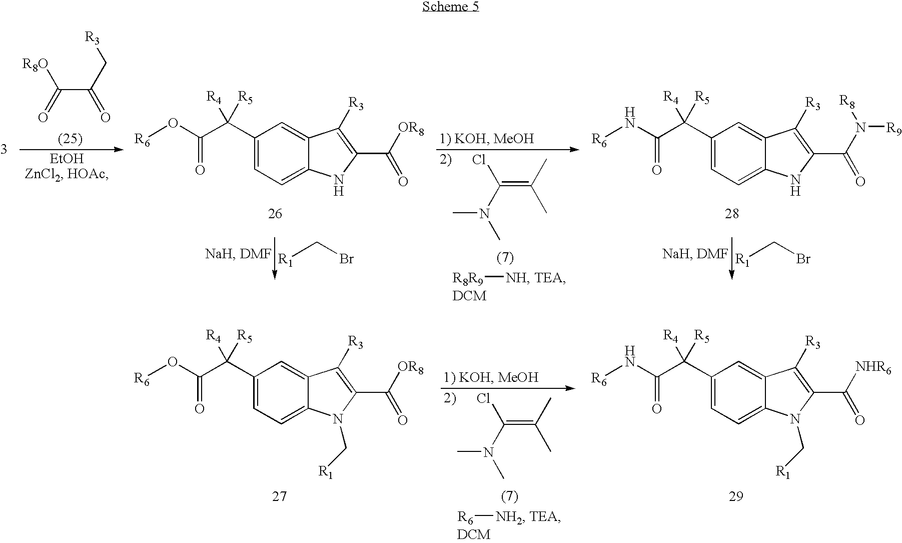

### Example 1

### Ethyl 2-[2-(3,5-dimethylphenyl)-1H-indol-5-yl]-2-methylpropanoate

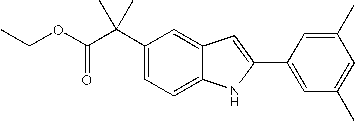

Step 1: Ethyl (4-nitrophenyl)acetate

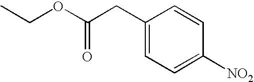

In a 12 L 3-neck RB flask (equipped with a thermocouple, stirring paddle, a condenser blanketed with a nitrogen line, and a heating mantle) were added 4-nitrophenylacetic acid (Aldrich) (500.00 g) and ethanol (4 L), followed by concentrated sulfuric acid (150 mL) (added slowly). The bright yellow reaction mixture was then heated to reflux for 2 hrs. The reaction was then allowed to stir at room temperature overnight. The reaction was concentrated under reduced pressure, and the residue (pale, yellow solid) obtained was stirred with hepatne to a thick slurry. The solid product was collected on the filter, washed with heptane, and dried in a vacuum oven to the give the desired product as a light-yellow solid (570 g).

¹H-NMR (CDCl₃): δ 1.24 (t, 3H), 3.75 (s, 2H), 4.18 (q, 2H), 7.48 (d, 2H), 8.20 (d, 2H)

Mass Spectra (m/e): 210.2 (M+H).

Step 2: Ethyl 2-methyl-2-(4-nitrophenyl)propanoate

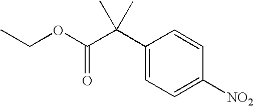

In a 22 L 3-neck RB flask (equipped with a claisen adapter with a thermocouple and a nitrogen line, stirring paddle, and an addition funnel covered by a septum) was placed anhydrous N,N-dimethylformamide (5.8 L) and cooled to 0° C. Sodium tert-butoxide (271 g), 97% (Aldrich) was then added in portions under stirring. After 30 min of stirring at 0° C., ethyl (4-nitrophenyl)acetate (570 g) (from Step 1 above) was added (in portions) to the reaction. To the resulting dark colored mixture was slowly added Iodomethane (175 mL), keeping the reaction temperature below +10° C. After 15 min of stirring, an additional Sodium tert-butoxide (271 g) was added in portions followed by an additional Iodomethane (175 mL), keeping the temp. below +10° C. all through. After 20 min of stirring, the process was repeated with the addition of an additional Sodium tert-butoxide (27 g) and Iodomethane (33 mL). The reaction was then slowly allowed to warm up to the room temperature overnight. The reaction mixture was poured into a mixture of ice-water (5 L), acetic acid (100 mL) and EtOAc (4 L), the layers were separated, and the aqueous was back extracted with EtOAc (4 L). The combined organic layers was washed with 0.5N aqueous HCl (1.2 L), dried over MgSO₄, filtered, and concentrated in vacuo to a dark, red oil (652 g).

¹H-NMR (CDCl₃): δ 1.20 (t, 3H), 1.62 (s, 6H), 4.18 (q, 2H), 7.50 (d, 2H), 8.20 (d, 2H)

Mass Spectra (m/e): 238.2 (M+H).

Step 3: Ethyl 2-(4-aminophenyl)-2-methylpropanoate

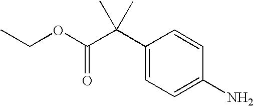

To a solution of ethyl 2-methyl-2-(4-nitrophenyl)-propanoate (652 g) (from Step 2, above) in ethanol (8L) was carefully added 10% Pd/C catalyst (40 g) under a stream of N2. The mixture was then stirred under hydrogen atmosphere at 40 psi at room temperature for 24 h. The reaction was filtered through a pad of celite, washed with EtOH, and the combined filtrate was concentrated in vacuo to give the desired product as oil (565 g; yellow).

¹H-NMR (CDCl₃): δ 1.20 (t, 3H), 1.62 (s, 6H), 4.18 (q, 2H), 6.66 (d, 2H), 7.20 (d, 2H)

Mass Spectra (m/e): 208 (M+H).

Step 4: Ethyl 2-(4-hydrazinophenyl)-2-methylpropanoate

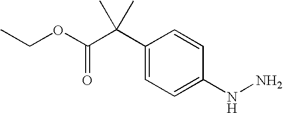

A mixture of ethyl 2-(4-aminophenyl)-2-methylpropanoate (562 g) (from Step 3 above) and concentrated hydrochloric acid (2.8 L) was placed in a 12 L 3-neck RB flask (equipped with a claisen adapter with a thermocouple and a nitrogen line, stirring paddle, and an addition funnel covered by a septum), and the mixture was stirred at room temperature for 1 h to provide a dark red/brown solution. The solution was then cooled to −10° C. and added an aqueous solution of sodium nitrite (203 g) in water (1.1 L) of water, keeping the reaction temperature between −5° C. and −10° C. The mixture was stirred at −10° C. for 30 min and then added slowly at −10° C. to a preformed solution of tin (II) chloride dihydrate (3065 g) in conc. HCl (2.2 L). After 1 hr at −10° C., the mixture was transferred into a large extractor containing water (8 L) and methyl t-butyl ether (8 L), and the reaction flask was rinsed with methyl t-butyl ether (4 L). The combined organic phase was separated, washed with H₂O and then treated with saturated sodium bicarbonate solution to pH=8 over night. The precipitated tin salt was removed by filtration. The organic phase from the filtrate was separated, washed with H₂O, dried over MgSO₄, filtered, and concentrated in vacuo to give the crude hydrazine as a dark-red oil (404 g). To a solution of the above crude hydrazine in dry ether (8 L) was added dropwise a 4.0M HCl in 1,4-dioxane (Aldrich) (500 mL) using an addition funnel. The resulting mixture was stirred overnight at room temperature and then diluted with heptane (3 L) and filtered. The solid product (orange solid) on the filter was washed with heptane and dried overnight in vacuo at 70° C. (233 g).

¹H-NMR (CDCl₃): δ 1.20 (t, 3H), 1.52 (s, 3H), 1.55 (s, 3H) 4.12 (q, 2H), 6.8 (d, 2H), 7.20 (d, 2H), 7.3 (m, 2H)

Mass Spectra (m/e): 223 (M+H).

Step 5: Ethyl 2-[2-(3,5-dimethylphenyl)-1H-indol-5-yl]-2-methylpropanoate

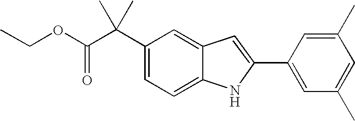

To a solution of ethyl 2-(4-hydrazinophenyl)-2-methylpropanoate (2.4 g) (from Step 4 above) and 3,5-dimethyl acetophenone (1.26 g) in AcOH (30 mL) was added anhydrous zinc chloride (3.5 g) at room temperature, and the resulting mixture was stirred at 100° C. for 16 hours. The reaction was then cooled to room temperature and concentrated in vacuo. The residue obtained was partitioned between ethylacetate (100 mL) and water (100 mL). The organic phase was separated and washed with saturated sodium bicarbonate and brine, then dried over sodium sulfate, filtered and concentrated in vacuo. The crude product thus obtained was purified by silica-gel chromatography using 20% EtOAC in hexanes to give the desired titled indole as solid.

¹H-NMR (CDCl₃): δ 1.20 (t, 3H), 1.6 (s, 6H), 2.43 (s, 6H), 4.12 (q, 2H), 6.8 (s, 1H), 7.0 (s, 1H) 7.18 (d, 1H), 7.30 (s, 1H), 7.33 (s, 1H), 7.38 (d, 1H), 7.64 (s, 1H), 8.20 (br s, 1H)

Mass Spectra (m/e): 336 (M+H).

### Example 2

### Ethyl 2-methyl-2-{2-[4-(trifluoromethoxy)phenyl]-1H-indol-5-yl}propanoate

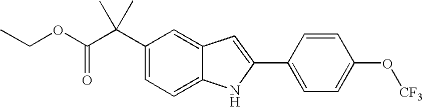

A solution of ethyl 2-(4-hydrazinophenyl)-2-methylpropanoate (0.4 g) (from Step 4, EXAMPLE 1) in EtOH (3 mL) was placed in a Biotage microwave reaction vial. To the solution were then added 4-(trifluoromethoxy)acetophenone (0.24 mL) and acetic acid (0.045 mL), and the mixture was heated under microwave at 110° C. for 30 min. After cooling the reaction to room temperature, anhydrous ZnCl₂ (0.38 g) was added. The reaction was then continued under microwave at 180° C. for 30 min. The reaction was then cooled to room temperature and partitioned between ethylacetate (100 mL) and water (100 mL). The organic phase was separated and washed with saturated sodium bicarbonate and brine, then dried over sodium sulfate, filtered and concentrated in vacuo. The crude product obtained was purified by silica-gel chromatography using 20% methyl t-butyl ether in heptane to give the titled indole as solid.

¹H-NMR (CDCl₃): δ 1.22 (t, 3H), 1.68 (s, 6H), 4.16 (q, 2H), 6.8 (s, 1H), 7.24 (d, 1H), 7.32 (d, 2H), 7.38 (d, 1H), 7.64 (s, 1H), 7.69 (d, 2H), 8.20 (br s, 1H)

Mass Spectra (m/e): 392 (M+H).

Using the procedures described in EXAMPLES 1 and 2 the following 2-substituted indoles were prepared.

| | | | |
|---|---|---|---|
| | | | Mass Spectral |
| | | | Data m/e |
| Example | Structure | Chemical Name | (M + H) |

| | | | |
|---|---|---|---|
| 3 | 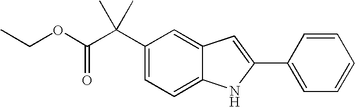 | Ethyl 2-methyl-2-(2- phenyl-1H-indol-5- yl)propanoate | 308 |
| 4 | 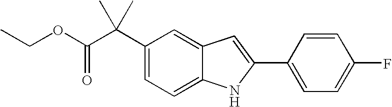 | Ethyl 2-[2-(4- fluorophenyl)-1H-indol- 5-yl]-2-methylpropanoate | 326 |
| 5 | 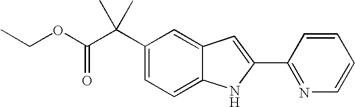 | Ethyl 2-methyl-2-(2- pyridin-2-yl-1H-indol-5- yl)propanoate | 309 |
| 6 | 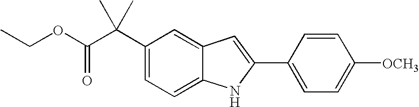 | Ethyl 2-[2-(4- methoxyphenyl)-1H- indol-5-yl]-2- methylpropanoate | 338 |
| 7 | 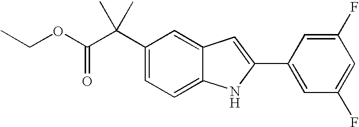 | Ethyl 2-[2-(3,5- difluorophenyl)-1H- indol-5-yl]-2- methylpropanoate | 344 |
| 8 | 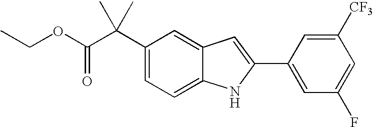 | Ethyl 2-methyl-2-{2-[3- methyl-5- (trifluoromethyl)phenyl]- 1H-indol-5-yl}propanoate | 394 |
| 9 | 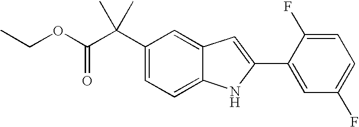 | Ethyl 2-[2-(2,5- difluorophenyl)-1H- indol-5-yl]-2- methylpropanoate | 344 |
| 10 | 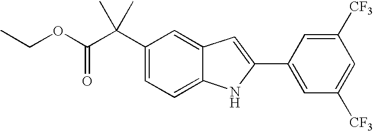 | Ethyl 2-[2-(3,5-bis- (trifluoromethyl)phenyl)- 1H-indol-5-yl]-2- methylpropanoate | 444 |
| 11 | 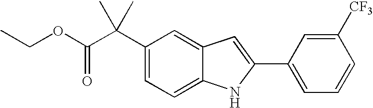 | Ethyl 2-[2-(3- (trifluoromethyl)phenyl)- 1H-indol-5-yl]-2- methylpropanoate | 376 |
| 12 | 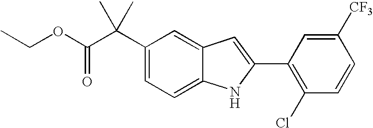 | Ethyl 2-[2-(2-chloro-5- (trifluoromethyl)phenyl)- 1H-indol-5-yl]-2- methylpropanoate | 410 |
| 13 | 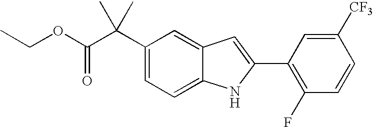 | Ethyl 2-[2-(2-fluoro-5- (trifluoromethyl)phenyl)- 1H-indol-5-yl]-2- methylpropanoate | 394 |
| 14 | 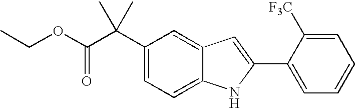 | Ethyl 2-methyl-2-{2-[2- (trifluoromethyl)phenyl]- 1H-indol-5-yl}propanoate | 376 |
| 15 | 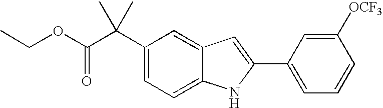 | Ethyl 2-methyl-2-{2-[3- (trifluoromethoxy)phenyl]- 1H-indol-5-yl}propanoate | 392 |
| 16 | 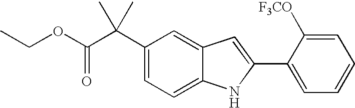 | Ethyl 2-methyl-2-{2-[2- (trifluoromethoxy)phenyl]- 1H-indol-5-yl}propanoate | 392 |
| 17 | 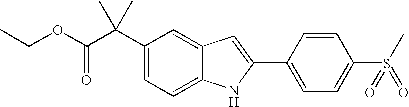 | Ethyl 2-methyl-2-{2-[4- (methylsulfonyl)phenyl]- 1H-indol-5-yl}propanoate | 386 |
| 18 | 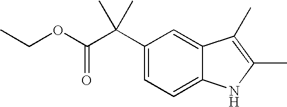 | Ethyl 2-(2,3-dimethyl- 1H-indol-5-yl)-2- methylpropanoate | 260 |

### Example 19

### 2-[2-(3,5-dimethylphenyl)-1H-indol-5-yl]-(N-cyclopropyl)-2-methylpropanamide

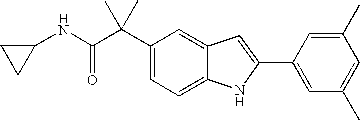

Step 1: 2-[2-(3,5-dimethylphenyl)-1H-indol-5-yl]-2-methylpropanoic acid

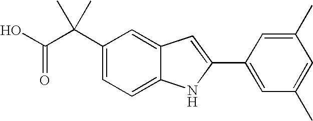

To a solution of ethyl 2-[2-(3,5-dimethylphenyl)-1H-indol-5-yl]-2-methylpropanoate (from Step 5, EXAMPLE 1) (1.5 g) in methanol (20 mL) was added aqueous 2M KOH (4 mL) at room temperature and the reaction was refluxed for 16 h. After cooling to room temprature, the reaction was concentrated in vacuo. The residue thus obtained was partitioned between EtOAc and 1N HCl. The organic phase was then washed with brine, then dried over sodium sulfate, filtered and concentrated in vacuo to give the titled product (1.2 g).

¹H-NMR (CDCl₃): 1.6 (s, 6H), 2.43 (s, 6H), 6.8 (s, 1H), 7.0 (s, 1H), 7.18 (d, 1H), 7.30 (s, 1H), 7.33 (s, 1H), 7.38 (d, 1H), 7.64 (s, 1H), 8.20 (br s, 1H)

Mass Spectra (m/e): 308 (M+H).

Step 2: 2-[2-(3,5-dimethylphenyl)-1H-indol-5-yl]-(N-cyclopropyl)-2-methylpropanamide

To a solution of 2-[2-(3,5-dimethylphenyl)-1H-indol-5-yl]-2-methylpropanoic acid (0.31 g) in acetonitrile (5 mL) was added 1-chloro-N,N-2-trimethylpropenylamine (0.14 mL) at 0° C. The mixture was then stirred at room temperature for 15 min and then concentrated in vacuo. The residue obtained was dissolved in methylene chloride (5 mL) and treated with cyclopropyl amine (0.2 mL) at room temperature for 30 min. The reaction was then partitioned between EtOAc (15 mL) and water (15 mL). The organic phase was separated and washed with 10% sodium biocarbonate solution, brine, then dried over sodium sulfate, filtered and concentrated in vacuo to give the titled product.

¹H-NMR (CDCl₃): δ 1.6 (s, 6H), 2.43 (s, 6H), 4.12 (q, 2H), 6.8 (s, 1H), 7.0 (s, 1H), 7.18 (d, 1H), 7.30 (s, 1H), 7.33 (s, 1H), 7.38 (d, 1H), 7.64 (s, 1H), 8.20 (br s, 1H)

Mass Spectra (m/e): 336 (M+H).

### Example 20

### 2-{7-bromo-1-[2-(tert-butylamino)-2-oxoethyl]-2-[4-(trifluoromethoxy)phenyl]-1H-indol-5-yl}-N-(tert-butyl)-2-methylpropionamide

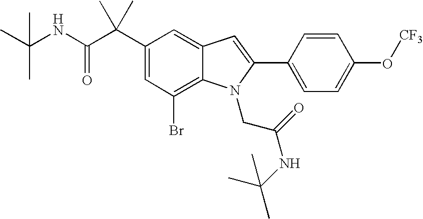

Step 1: Ethyl 2-(4-amino-3-bromo-5-iodophenyl)-2-methylpropanoate

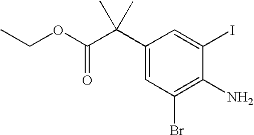

To a solution of ethyl 2-(4-amino-3-iodophenyl)-2-methylpropanoate (prepared as described by Walsh et. al. in Tetrahedron 2001, 57, 5233-5241) (2.0g, 5.62 mMol) in dry THF (60 mL) was slowly added a solution of Pyridinium bromide perbromide (2.698 g, 8.44 mmol) in THF (60 ml) under stirring at room temperature. After stirring for 45 minutes, the mixture was filtered and partitioned between ethylacetate and 10% NaHSO₃. The organic phase was washed with saturated sodium bicarbonate, and brine, then dried over sodium sulfate, filtered and concentrated. The residue obtained was purified by column chromatography on silica gel Biotage 25M, eluting with EtOAc/isohexane to give the titled product as brown solid.

Mass Spectra (m/e): 399 (M+H).

Step 2: Trimethyl {[4-(trifluoromethoxy)phenyl]ethynyl}silane

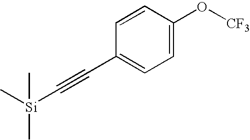

To a solution of 1-iodo-4-(trifluoromethoxy)benzene (1.087 mL, 6.94 mMol) and TMS-acetylene (1.166 ml, 8.33 mmol) in THF (30ml) were added Copper(I) iodide (0.066 g, 0.347 mMol), Trans-Bis(triphenylphosphine)palladium(II)chloride (0.244 g, 0.347 mmol) and triethylamine (2.90 ml, 20.83 mmol). After stirring for 3 h at room temperature, the reaction mixture was concentrated. The residue was dissolved in heptane, filtered through a plug of silica gel and concentrated to give the desired product as oil.

Mass Spectra (m/e): 259 (M+H)

Step 3: 1-Ethynyl-4-(trifluoromethoxy)benzene

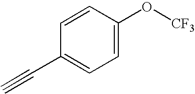

To a solution of trimethyl{[4-(trifluoromethoxy)phenyl]ethynyl}silane (0.89 g, 3.45 mMol) (from Step 2) in THF (8 mL) was added TBAF (3.79 mL, 3.79 mMol) slowly. After stirring for 1 h at room temperature, the reaction was concentrated and partitioned between dichloromethane and water. The organic phase was washed with water, dried over magnesium sulfate, filtered and concentrated to give the desired product.

Mass Spectra (m/e): 187 (M+H)

Step 4: Ethyl 2-(4-amino-3-bromo-5-{[4-(trifluoromethoxy)phenyl]ethynyl}phenyl)-2-methylpropanoate

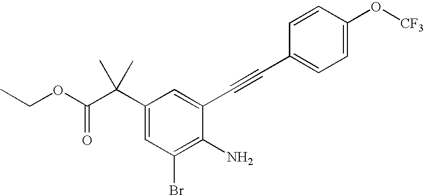

To a solution of 1-ethynyl-4-(triflubromethoxy)benzene (0.415 g, 2.23. mMol) (from Step 3) and ethyl 2-(4-amino-3-bromo-5-iodophenyl)-2-methylpropanoate (0.89 g, 2.23 mMol) (from Step 1) in THF (9 mL) were added Copper(I) iodide (0.021 g, 0.110 mMol), trans-bis(triphenylphosphine)palladiumchloride (0.077 g, 0.110 mMol) and triethylamine (0.923 mL, 6.62 mMol). After stirring for 3 h at room temperature, the reaction was concentrated and the crude product obtained was purified by silica-gel chromatography using methyl t-butylether in heptane (gradient 0-40%).

Mass Spectra (m/e): 470 (M+H).

Step 5: Ethyl 2-{7-bromo-2-[4-(trifluoromethoxy)phenyl]-1H-indol-5-yl}-2-methylpropanoate

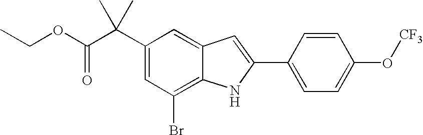

To a solution of ethyl 2-(4-amino-3-bromo-5-{[4-(trifluoromethoxy)phenyl]ethynyl}phenyl)-2-methylpropanoate (0.934 g, 1.988 mmol) (from Step 4) in N-Methyl-2-pyrrolidinone (9.00 ml) was added dropwise a solution of potassium t-butoxide (0.468 g, 4.17 mmol) in N-Methyl-2-pyrrolidinone (9 ml) at room temperature. After stirring at that temperature for 4 h, the reaction was quenched with water and extracted with methyl-butylether. The organic phase was dried over sodium sulfate, filtered and concentrated. The residue obtained was purified by column chromatography on silica gel methyl t-butylether in heptane (gradient 0-40%).

Mass Spectra (m/e): 470 (M+H).

Step 6: Ethyl 2-{7-bromo-1-[2-(tert-butylamino)-2-oxoethyl]-2-[4-(trifluoromethoxy)phenyl]-1H-indol-5-yl}-2-methylpropanoate

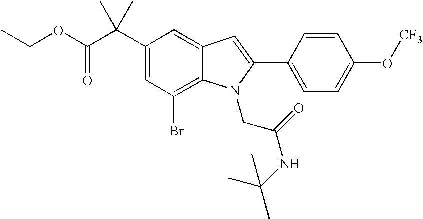

To a stirred suspension of NaH (0.028 g) in DMF (1 mL) was added ethyl 2-{7-bromo-2-[4-(trifluoromethoxy)phenyl]-1H-indol-5-yl}-2-methylpropanoate (0.252 g, 0.537 mmol) (from Step 5) in DMF (1 ml) at 0° C. After stirring at room temperature for 45 min, the reaction was cooled to 0° C., added 2-bromo-N-(tert-butyl)acetamide (0.23 g, 1.181 mmol) and stirred at room temperature for 12 h. The reaction mixture was then diluted with ethylacetate and washed with water and brine, then dried over magnesium sulfate, filtered and concentrated. The crude product was purified by HPLC Reverse phase (C-18) using acetonitrile/water +0.1% TFA gradient to afford the titled product.

Mass Spectra (m/e): 583 (M+H).

Step 7: 2-{7-bromo-1-[2-(tert-butylamino)-2-oxoethyl]-2-[4-(trifluoromethoxy)phenyl]-1H-indol-5-yl}-2-methylpropionic acid

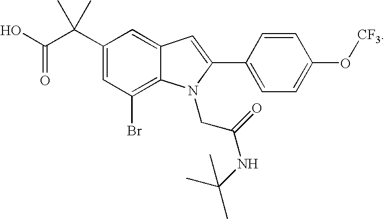

To a solution of Ethyl 2-{7-bromo-1-[2-(tert-butylamino)-2-oxoethyl]-2-[4-(trifluoromethoxy)phenyl]-1H-indol-5-yl}-2-methylpropanoate (0.33 g, 0.56 mMol) (from Step 6) in MeOH (6 mL) was added aqueous 2M KOH (0.578 ml, 1.156 mMol) was added, and the mixture was stirred at 85° C. for 16 hours. The reaction was cooled to room temperature, concentrated and partitioned between ether and 2N NaOH. The aqueous layer was acidified with 1N HCl, and extracted with ethylacetate. The organic phase was washed with brine, dried (Na₂SO₄)and concentrated to give the desired product.

Mass Spectra (m/e): 555 (M+H).

Step 8: 2-{7-bromo-1-[2-(tert-butylamino)-2-oxoethyl]-2-[4-(trifluoromethoxy)phenyl]-1H-indol-5-yl}-N-(tert-butyl)-2-methylpropionamide

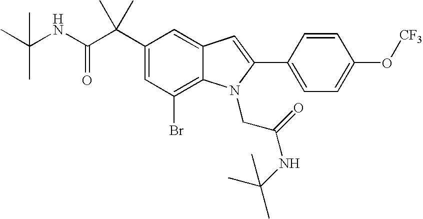

To a solution of 2-{7-bromo-1-[2-(tert-butylamino)-2-oxoethyl]-2-[4-(trifluoromethoxy)phenyl]-1H-indol-5-yl}-2-methylpropionic acid (0.277 g, 0.499 mMol) (from Step 7) in CH₂Cl₂ (3 ml) was added 1-chloro-N,N-2-trimethylpropenylamine (0.073 mL, 0.549 mMol) at 0° C. After stirring for 45 min at room, a mixture of t-butylamine (0.079 ml, 0.748 mmol) and triethylamine (0.104 mL, 0.748 mmol) in CH₂Cl₂ (5 ml) were added slowly at 0° C. After 6 h of stirring at room temperature, the reaction was diluted with ethylacetate, washed with saturated sodium bicarbonate and brine. The organic phase was concentrated and the residue obtained was purified by column chromatography on silica gel Biotage 40M using EtOAc/hexanes as the eluent to give the titled product (0.12 g).

Mass Spectra (m/e): 610 (M+H).

EXAMPLES 21-34 (Table 1) were prepared employing the known procedures described for the synthesis of similar compounds in the following publications. [(a). Walsh et. al. in Tetrahedron 2001, 57, 5233-5241 (b) Chu et. al. Tetrahedron Lett. 1997, 38, 3871-3874 (c) Ishiyama et. al. Tetrahedron Lett. 1997, 38, 3447-3450 (d) Giroux et.al. Tetrahedron Lett. 1997, 38, 3841-3844 (e) Larock et. al. J. Amer. Chem Soc. 1991, 113,6689-6690 (f) Chen et. al. Tetrahedron Lett. 1994, 35, 6981-6984].

TABLE 1

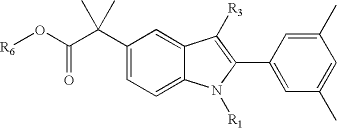

| | | | | Mass |
|---|---|---|---|---|
| | | | | Spectral |
| | | | | Data m/e |
| Example | R₁ | R₃ | R₆ | (M + H) |
| 21 | —COOt-Bu | —CH(CH₃)CH₂OH | Et | 494 |
| 22 | —COOt-Bu | —CH(CH₃)CH₂OCH₂Ph | Et | 584 |
| 23 | —COOt-Bu | —CH₂CH₂OH | Et | 480 |
| 24 | —COOt-Bu | —CH(CH₃)CH₂NHCOCH₂O—COOtBu | Et | 651 |
| 25 | —COOt-Bu | —CH(CH₃)CH₂NHCOCH₂OH | Et | 551 |
| 26 | —COOt-Bu | —CH(CH₃)CH₂NHCOC(c-Pr)OH | Et | 577 |
| 27 | —COPh(4-Cl) | —CH(Ph)CH₂OH | Me | 580 |
| 28 | —CH₂CH(OH)CH₃ | —CH₂CH₂OH | Et | 438 |
| 29 | —COOt-Bu | —CH(CH₃)CH₂NH₂ | Et | 493 |
| 30 | —COOt-Bu | —CH(CH₃)CH₂N(CH₃)SO₂CH₃ | Et | 585 |
| 31 | —CH₃ | —CH(CH₃)CH₂N(CH₃)SO₂CH₃ | Et | 499 |
| 32 | —COOt-Bu | —CH(CH₃)CH₂NHCH₃ | Et | 507 |
| 33 | —COOt-Bu | —CH(CH₃)CH₂NHCOC(c-Pr)OCOOtBu | Et | 677 |
| 34 | —COOt-Bu | —CH(CH₃)CH₂NHCOC(Et)₂OCOOtBu | Et | 707 |

EXAMPLES 35-95 (Table 2-4) were synthesized using the procedures and intermediates described above in EXAMPLES 1-20.

TABLE 2

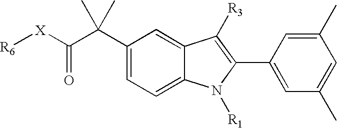

| | | | | | Mass |
|---|---|---|---|---|---|
| | | | | | Spectral |
| | | | | | Data m/e |
| Example | R₁ | R₃ | X | R₆ | (M + H) |
| 35 | —CH₂CH(OH)CH₃ | H | NH | c-Propyl | 405 |
| 36 | —CH₂CH(OH)CH₃ | H | NH | CF₃CH₂— | 447 |
| 37 | —CH₂CH(OH)CH₂F | H | NH | t-Bu | 439 |
| 38 | —CH₂CH(OH)CH₂OH | H | NH | t-Bu | 437 |
| 39 | —CH₂CH(OH)CH₂Ot-Bu | H | NH | t-Bu | 493 |
| 40 | —CH₂CH(OH)Ph | H | NH | t-Bu | 483 |
| 41 | —CH₂CH(OH)CH₃ | H | NH | t-Bu | 421 |
| 42 | —COOt-Bu | H | NH | t-Bu | 463 |
| 43 | —COPh(4-Cl) | H | NH | t-Bu | 501 |
| 44 | —CH₂Ph(4-Cl) | H | NH | t-Bu | 487 |
| 45 | —CH₂CH₂OCH₃ | H | NH | t-Bu | 421 |
| 46 | —CH₂CONHtBu | H | NH | t-Bu | 476 |
| 47 | —CH₂COOtBu | H | NH | t-Bu | 477 |
| 48 | —CH₂CH₂OH | H | NH | t-Bu | 407 |
| 49 | —CH₂CH₂CH₂OH | H | NH | t-Bu | 421 |
| 50 | —CH₂CONH-c-Pr | H | NH | t-Bu | 460 |
| 51 | —CH₂CH₂CH₂OCH₃ | H | NH | t-Bu | 435 |
| 52 | 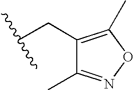 | 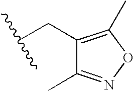 | NH | t-Bu | 581 |
| 53 | 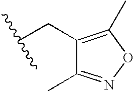 | H | NH | tBu | 472 |
| 54 | —CH₂CONHtBu | —CH₂CONHtBu | NH | H | 533 |
| 55 | —CH₂CONHtBu | H | NH | H | 420 |
| 56 | —CH₂CH(OH)CH₃ | H | N-Me | Me | 393 |

TABLE 3

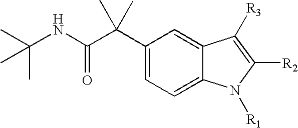

| | | | | Mass |
|---|---|---|---|---|
| | | | | Spectral |
| | | | | Data m/e |
| Example | R₁ | R₂ | R₃ | (M + H) |
| 57 | —CH₂CONH-t-Bu | CH₃ | CH₃ | 400 |
| 58 | —CH₂CONH-t-Bu | 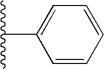 | H | 448 |
| 59 | —CH₂CONH-t-Bu | 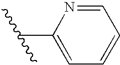 | H | 449 |
| 60 | —CH₂CONH-t-Bu | 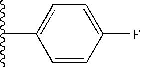 | H | 466 |
| 61 | —CH₂CH(OH)CH₃ | 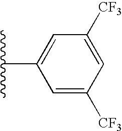 | H | 529 |
| 62 | —CH₂CH(OH)CH₃ | 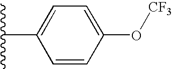 | H | 477 |
| 63 | —CH₂CONH-t-Bu | 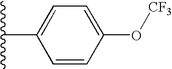 | H | 532 |
| 64 | —CH₂CONHtBu | 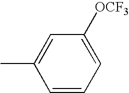 | H | 532 |
| 65 | —CH₂CONH-t-Bu | 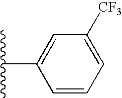 | H | 516 |
| 66 | —CH₂CH(OH)CH₃ | 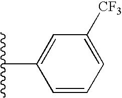 | H | 461 |
| 67 | —CH₂CONH-t-Bu | 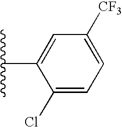 | H | 551 |
| 68 | —CH₂CH(OH)CH₃ | 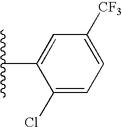 | H | 495 |
| 69 | —CH₂CH(OH)CH₃ | 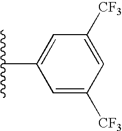 | CH₃ | 543 |
| 70 | 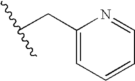 | 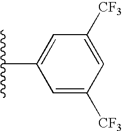 | H | 676 |
| 71 | —CH₂CONH-t-Bu | 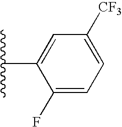 | H | 534 |
| 72 | —CH₂CONH-t-Bu | 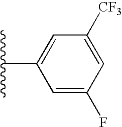 | H | 534 |
| 73 | —CH₂CONH-t-Bu | 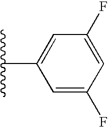 | H | 484 |
| 74 | —CH₂CONH₂ | 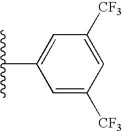 | H | 528 |
| 75 | —CH₂CONHtBu | 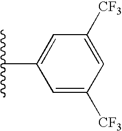 | H | 584 |
| 76 | 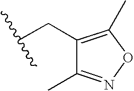 | 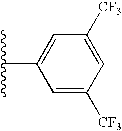 | H | 580 |
| 77 | 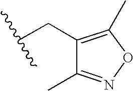 | 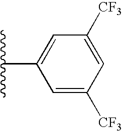 | 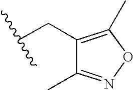 | 689 |
| 78 | 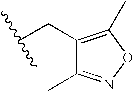 | 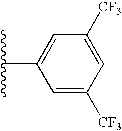 | CH₃ | 594 |
| 79 | —CH₂CH₂OH | 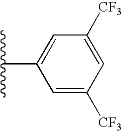 | CH₃ | 529 |
| 80 | —CH₂CH₂OH | 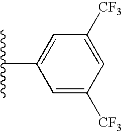 | H | 515 |
| 81 | —CH₂CH₂CH₂OH | 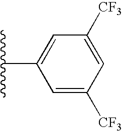 | H | 529 |
| 82 | 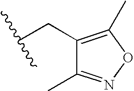 | 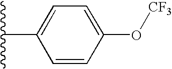 | 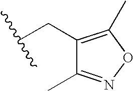 | 637 |
| 83 | 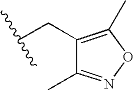 | 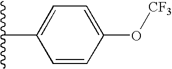 | H | 528 |
| 84 | —CH₂CONHtBu | 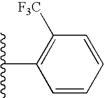 | —H | 516 |
| 85 | —CH₂CONHtBu | 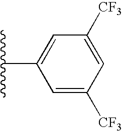 | CH₃ | 598 |
| 86 | —CH₂CH(OH)CH₃ | 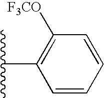 | H | 477 |
| 87 | —CH₂CH₂OH | 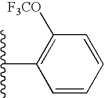 | H | 463 |
| 88 | —CH₂CONH-t-Bu | 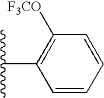 | H | 532 |
| 89 | 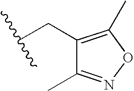 | 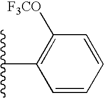 | H | 528 |
| 90 | 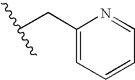 | 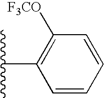 | H | 510 |
| 91 | —CH₂COOMe | 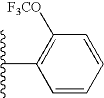 | H | 491 |
| 92 | —CH₂CONH₂ | 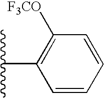 | H | 476 |
| 93 | —CH₂CONH-t-Bu | 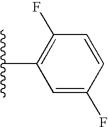 | H | 484 |
| 94 | —CH₂CONH-t-Bu | 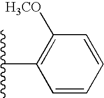 | H | 478 |
| 95 | 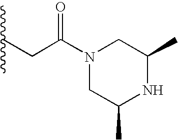 | 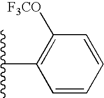 | H | 573 |
| 96 | 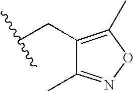 | CH₃ | CH₃ | 396 |
| 97 | —CH₂CH(OH)CH₃ | CH₃ | CH₃ | 345 |
| 98 | 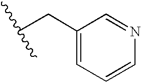 | CH₃ | CH₃ | 378 |
| 99 | 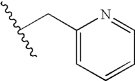 | CH₃ | CH₃ | 378 |
| 100 | 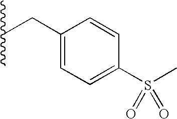 | 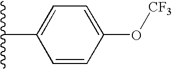 | H | 587 |
| 101 | 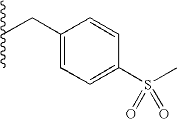 | 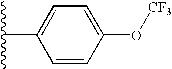 | 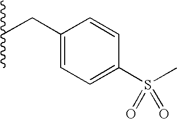 | 755 |
| 102 | CH₃ | 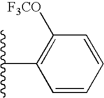 | H | 433 |
| 103 | —CH₂COOH | 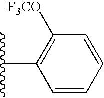 | H | 477 |

TABLE 4

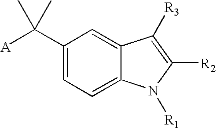

| | | | | | Mass |
|---|---|---|---|---|---|
| | | | | | Spectral |
| | | | | | Data m/e |
| Example | A | R₁ | R₂ | R₃ | (M + H) |
| 104 | —CH₂OH | —COOt-Bu | 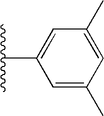 | —CH(CH₃)CH₂NHCOOtBu | 551 |
| 105 | —CH₂OH | —CH₂CONHt-Bu | 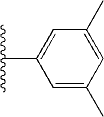 | —CH₂CONHtBu | 520 |
| 106 | —CH₂OH | —CH₂CONHt-Bu | 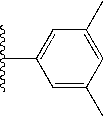 | H | 407 |
| 107 | —CH₂OH | —CH₂CH(OH)CH₃ | 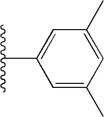 | H | 352 |
| 108 | —COOEt | —CH₂CONHt-Bu | 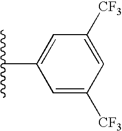 | CH₃ | 571 |
| 109 | —COOH | —CH₂CONHt-Bu | 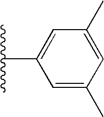 | H | 421 |
| 110 | —CONH-c-Pr | —CH₂CONHt-Bu | 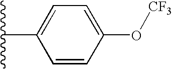 | H | 516 |
| 111 | —CONH₂ | —CH₂CONHt-Bu | 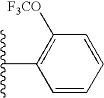 | H | 476 |
| 112 | —CON(CH₃)₂ | —CH₂CONHt-Bu | 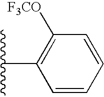 | H | 504 |
| 113 | 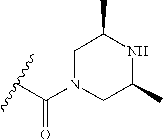 | —CH₂CONHt-Bu | 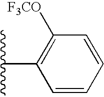 | H | 573 |

The following 2-substituted indoles (Table 5) were prepared by employing the procedures described in EXAMPLES 1 and 2.

TABLE 5

| | | | Mass |
|---|---|---|---|
| | | | Spectral |
| | | | Data m/e |
| Example | Structure | Chemical Name | (M + H) |
| 114 | 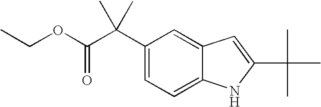 | Ethyl 2-(2-tert-butyl-1H- indol-5-yl)-2- methylpropanoate | 288 |
| 115 | 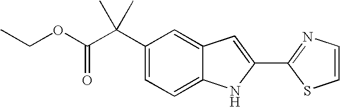 | Ethyl 2-methyl-[2-(1,3- thiazol-2-yl]-1H-indol-5- yl)-propanoate | 315 |

### Example 116

### N-(tert-butyl)-5-[2-(tert-butylamino)-1,1-dimethyl-2-oxoethyl]-1-[2-[tert-butylamino-2-oxoethyl]-1H-indol-2-carboxamide

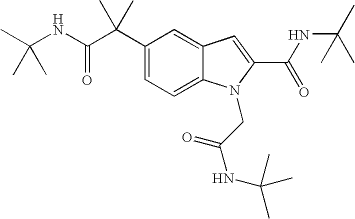

Step 1: Ethyl 5-(2-ethoxyl-1,1-dimethyl-2-oxoethyl)-1H-indole-2-carboxylate:

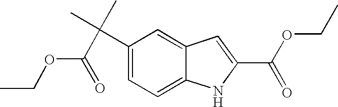

A solution of ethyl 2-(4-hydrazinophenyl)-2-methylpropanoate (0.85 g, 3.29 mmol) (from Step 4, EXAMPLE 1) in EtOH (2 mL) was placed in a Biotage microwave reaction vial. To the solution were then added ethyl pyruvate (0.36 mL, 3.29 mmol)) and acetic acid (0.094 mL, 1.64 mmol), and the mixture was heated under microwave at 120° C. for 30 min. After cooling the reaction to room temperature, anhydrous ZnCl₂ (1343 mg, 9.86 mmol) was added. The reaction was then continued under microwave at 180° C. for 30 min. The reaction was then cooled to room temperature and partitioned between ethylacetate (100 mL) and water (100 mL) The organic phase was separated and washed with saturated sodium bicarbonate and brine, then dried over sodium sulfate, filtered and concentrated in vacuo. The crude product obtained was purified by silica-gel chromatography using 20% methyl t-butyl ether in heptane to give the titled indole as solid. (0.995 g)

Mass Spectra (m/e): 305 (M+H).

Step 2: :Ethyl 1-(2-tert-butylamino)-2-oxoethyl-5-(2-ethoxyl-1,1-dimethyl-2-oxoethyl)-1H-indole-2-carboxylate:

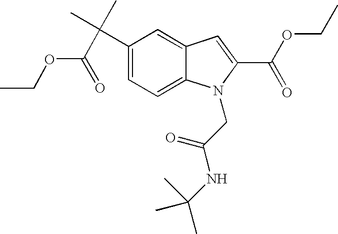

To a stirred suspension of NaH (0.06 g) in DMF (1 mL) was added ethyl 5-(2-ethoxyl-1,1-dimethyl-2-oxoethyl)-1H-indole-2-carboxylate (0.425 g, 1.40 mmol) (from Step 1) in DMF (1 ml) at 0° C. After stirring at room temperature for 45 min, the reaction was cooled to 0° C., added 2-bromo-N-(tert-butyl)acetamide (0.408 g, 2.10 mmol) and stirred at room temperature for 12 h. The reaction mixture was then diluted with ethylacetate and washed with water and brine, then dried over magnesium sulfate, filtered and concentrated to give the crude product (0.47 g)

Mass Spectra (m/e): 417 (M+H).

Step 3: 1-[2-(tert-butylamino)-2-oxoethyl]-5-(1-carboxy-1-methylethyl)-1H-indole-2-carboxylic acid

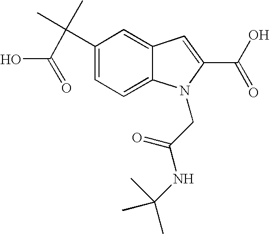

To a solution of ethyl 1-(2-tert-butylamino)-2-oxoethyl-5-(2-ethoxyl-1,1-dimethyl-2-oxoethyl)-1H-indole-2-carboxylate (0.75 g, 1.80 mmol) (from Step 2) in MeOH (5 mL) was added aqueous 2M KOH (1.80 mL, 3.60 mmol) was added, and the mixture was stirred at 85° C. for 16 hours. The reaction was cooled to room temperature, concentrated and partitioned between ether and 2N NaOH. The aqueous layer was acidified with

The aqueous layer was acidified with 1N HCl, and extracted with ethylacetate. The organic phase was washed with brine, dried (Na₂SO₄) and concentrated to give the desired product (0.63 g).

Mass Spectra (m/e): 361(M+H).

Step 3: N-(tert-butyl)-5-[2-(tert-butylamino)-1,1-dimethyl-2-oxoethyl]-1-[2-[tert-butylamino-2-oxoethyl]-1H-indol-2-carboxamide

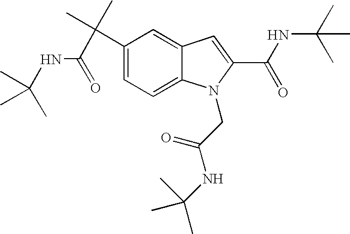

To a solution of 1-[2-(tert-butylamino)-2-oxoethyl]-5-(1-carboxy-1-methylethyl)-1H-indole-2-carboxylic acid (0.173 g, 0.48 mmol) in dichloromethane (1 mL) was added 1-chloro-N,N-2-trimethylpropenylamine (0.133 mL, 1 mmol) at 0° C. The mixture was then stirred at room temperature for 45 min and then cooled to 0° C. A mixture of t-butylamine (0.152 mL mL, 1.4 mmol) and triethylamine (0.1 mL, 0.72 mmol) in dichloromethane (5 mL) was added slowly to the reaction mixture and the solution was stirred at room temperature for 4 hours. The reaction was then partitioned between EtOAc and water. The organic phase was separated and washed with saturated sodium biocarbonate solution, brine, then dried over sodium sulfate, filtered and concentrated in vacuo to give the crude product. The residue was purified by column chromatography) on silica) gel Biotage 40M, eluting with EtOAc/Hexane to give the title product as a yellow solid.(0.116 g).

¹H-NMR (CDCl₃): δ 1.23 (s, 9H), 1.290 (s, 9H), 1.51 (s, 9H), 1.62 (s, 6H), 4.93 (s, 2H), 4.96 (br s, 1H), 6.15 (br s, 1H), 6.81 (s, 1H), 7.19 (br s, 1H), 7.31 (d, 1H), 7.56 (d, 1H), 7.60 (s, 1H).

Mass Spectra (m/e): 471 (M+H).

### Example 117

### N-(tent-butyl)-2-{1-[2-(tert-butylamino)-2-oxoethyl]-3-cyano-2-[4-(trifluoromethoxy)phenyl]-1H-indol-5-yl}-2-methylpropanamide

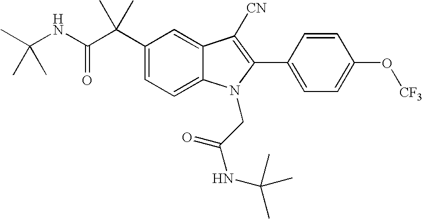

Chlorosulfonyl isocyanate (0.065 mL, 0.752 mmol) was added to a solution of N-(tert-butyl)-2-{1-[2-(tert-butylamimo)-2-oxoethyl}-2-[4-(trifluoromethoxyl)phenyl}-1H-indol-5-yl}-2-methylpropanamide (0.40 g, 0.752 mmol) (from Example 63) in acetonitrile (2 7 mL) at 0° C. over a period of 5 min and stirred for 30 min . Dry DMF (0.064 mL) was then added and the solution was heated under microwave at 150° C. for 1500 secs. The solution was partitioned between ethyl acetate and water, washed with saturated sodium bicarbonate, brine, dried over sodium sulfate. It was then filtered and concentrated to give the crude product. The residue was purified by preparative HPLC Reverse phase (C-18), eluting with Acetonitrile/Water +0.1% TFA, to give the titled product (0.235 mg) as a colorless solid.

¹H-NMR (CDCl₃): δ 1.28 (s, 9H), 1.34 (s, 9H), 1.62 (s, 6H), 4.54 (s, 2H), 5.07 (br s, 1H), 5.48 (br s, 1H), 7.31 (d, 1H), 7.38 (d, 1H), 7.43 (d, 2H), 7.65 (d, 2H), 7.77 (s, 1H).

Mass Spectra (m/e): 557 (M+H).

### Example 118

### N-(tert-butyl)-2-{1-[2-(tert-butylamino)-2-oxoethyl]-3-(methylthio)-2-[4-(trifluoromethoxy)phenyl]-1H-indol-5-yl}-2-methylpropanamide

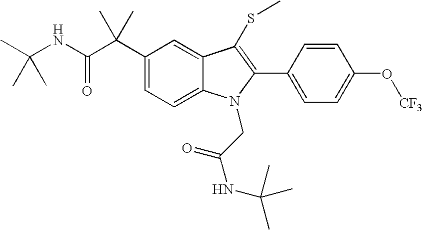

Magnesium bromide (3.46 mg, 0.019 mmol) was added to a solution of N-(methylthio)phthalimide (0.08 g, 0.414 mmol) and N-(tert-butyl)-2-{1-[2-(tert-butylamimo)-2-oxoethyl}-2-[4-(trifluoromethoxyl)phenyl}-1H-indol-5-yl}-2-methylpropanamide (Example 63) (200 mg, 0.376 mmol) in N,N′-dimethylacetamide (0.5mL) and the solution was heated under microwave at 150° C. for 1500 secs. After cooling the reaction to room temperature, the solution was partitioned between ethyl acetate and water, washed with satutated sodium biocarbonate solution, brine, then dried over sodium sulfate, filtered and concentrated in vacuo to give the crude product. The residue was purified by preparative HPLC Reverse phase (C-18), eluting with Acetonitrile/Water +0.1% TFA, to give the titled product (168 mg) as a colorless solid.

¹H-NMR (CDCl₃): δ 1.27 (s, 18H), 1.60 (s, 6H), 2.25 (s, 3H), 4.55 (s, 2H), 5.04 (br s, 1H), 5.19 (br s, 1H), 7.30-7.35 (m, 2H), 7.39 (d, 2H), 7.52 (d, 2H), 7.84 (s, 1H).

Mass Spectra (m/e): 578 (M+H).

### Example 119

### N-(tert-butyl)-2-{1-[2-(tert-butylamino)-2-oxoethyl]-3-(methylsulfonyl)-2-[4-(trifluoromethoxy)phenyl]-1H-indol-5-yl}-2-methylpropanamide

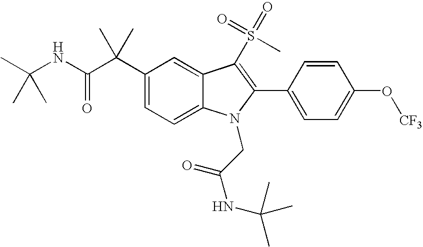

To a solution of N-(tert-butyl)-2-{1-[2-(tert-butylamino)-2-oxoethyl]-3-(methylthio)-2-[4-(trifluoromethoxy)phenyl]-1H-indol-5-yl}-2-methylpropanamide (0.097 g, 0.168 mmol) (Example 118) in methanol (1.2 mL) oxone (0.206 g, 0.336 mmol) was added and the reaction mixture was stirred at room temperature for 16 hours. The reaction mixture was partitioned between ethyl acetate and water, washed with satutated sodium bicarbonate solution, brine, then dried over sodium sulfate, filtered and concentrated in vacuo to give the crude product. The residue was purified by preparative HPLC Reverse phase (C-18), eluting with Acetonitrile/Water +0.1% TFA, to give the titled compound as a colorless solid (50 mg).

¹H-NMR (CDCl₃): δ 1.29 (s, 9H), 1.32 (s, 9H), 1.62 (s, 6H), 2.9 (s, 3H), 4.30 (s, 2H), 5.14 (br s, 1H), 5.41 (br s, 1H), 7.30 (s, 1H), 7.36-7.38 (m, 3H), 7.57 (d, 2H), 8.17 (s, 1H).

Mass Spectra (m/e): 610 (M+H).

### Example 120

### N-(tert-butyl)-2-{1-[2-(tert-butylamino)-2-oxoethyl]-3-(methylsulfinyl)-2-[4-(trifluoromethoxy)phenyl]-1H-indol-5-yl}-2-methylpropanamide:

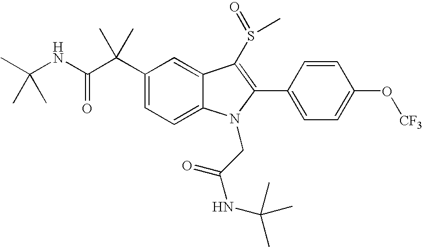

The titled compound was isolated as a solid (50 mg) from the reaction described in EXAMPLE 119. The product was formed as a result of an incomplete oxidation of the thiol compound described in EXAMPLE 118.

¹H-NMR (CDCl₃): δ 1.29 (s, 9H), 1.33 (s, 9H), 1.63 (d, 6H), 3.08 (s, 3H), 4.50 (s, 2H), 5.10 (br s, 1H), 5.43 (br s, 1H), 7.34-7.40 (m, 4H), 7.51 (d, 2H), 8.24 (s, 1H).

Mass Spectra (m/e): 594 (M+H).

Using the procedures described in EXAMPLES above the following compounds described in Table 6 were prepared.

TABLE 6

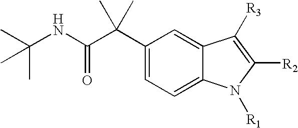

| | | | | Mass |
|---|---|---|---|---|
| | | | | Spectral |
| | | | | Data m/e |
| Example | R₁ | R₂ | R₃ | (M + H) |
| 121 | —CH₂CONH-t-Bu | 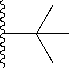 | H | 428 |
| 122 | 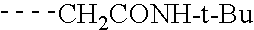 | 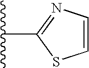 | H | 455 |
| 123 | —CH₂CONH-t-Bu | 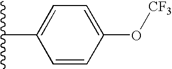 | CO₂H | 557 |
| 124 | —CH₂CONH-t-Bu | 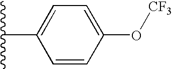 | CONH₂ | 557 |
| 125 | —CH₂CONH-t-Bu | 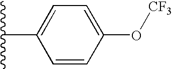 | SPh | 640 |
| 126 | —CH₂CONH-t-Bu | 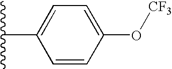 | S(O)Ph | 656 |
| 127 | —CH₂CONH-t-Bu | 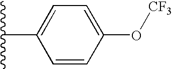 | SO₂Ph | 672 |
| 128 | —CH₂CONH-t-Bu | 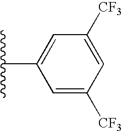 | SO₂Me | 662 |
| 129 | —CH₂CONH-t-Bu | 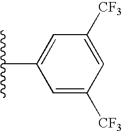 | SO₂Ph | 724 |
| 130 | 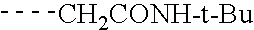 | 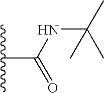 | CN | 496 |
| 131 | 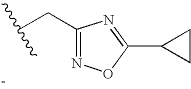 | 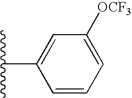 | H | 541 |
| 132 | —CH₂CONH-t-Bu | 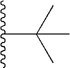 | H | 428 |
| 133 | 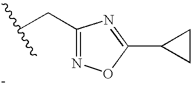 | 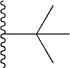 | H | 437 |
| 134 | 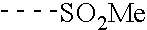 | 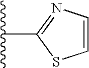 | H | 420 |
| 135 | —SO₂Me | 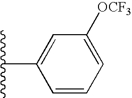 | —COMe | 539 |
| 136 | —CH₂CONH-t-Bu | 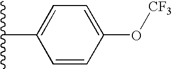 | 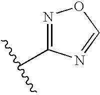 | 600 |
| 137 | —CH₂CONH-t-Bu | 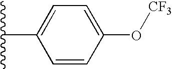 | 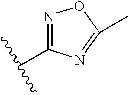 | 614 |
| 138 | —CH₂CONH-t-Bu | 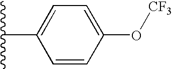 | 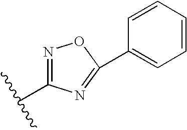 | 676 |
| 139 | —CH₂COOH | 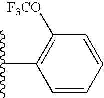 | H | 477 |

### Example 140

### N-(tert-butyl)-2-methyl-2-{1-pyridin-2-yl)-2-[4-(trifluoromethoxy)phenyl]-1H-indol-5-yl}propanamide

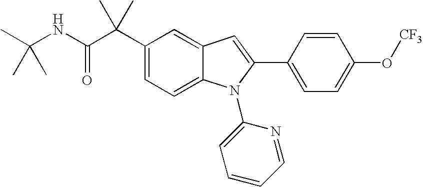

To a solution of N-(tert-butyl)-2-methyl-2-{1-pyridin-4-yl)-2-[4-(trifluoromethoxy)phenyl]-1H-indol-5-yl}propanamide (EXAMPLE 63) (0.041 g, 0.199 mmol) in toluene (0.3 mL) was added copper (I) iodide (0.023 g, 0.122 mmol), 2-iodopyridine (0.041 g, 0.199 mmol), N,N′-dimethylethylenediamine (0.026 mL, 0.245 mmol), potassium phosphate (0.143 g, 0.673 mmol) and the solution was heated in a sealed tube for 16 hours. After cooling to room temperature, the reaction mixture was partitioned between ethyl acetate and water, the organic phase was then washed with saturated sodium bicarbonate, brine, dried over sodium sulfate, filtered and concentrated in vacuo to give crude product. The residue was purified by HPLC Reverse phase (C-18), eluting with Acetonitrile/water +0.1% TFA to give the title product as a colorless solid. (25 mg).

¹-NMR (CDCl₃): 1.26 (s, 9H), 1.63 (s, 6H), 5.05 (br s, 1 H), 6.84 (s, 1H), 7.02 (d, 1H), 7.16 (d, 2H), 7.21(d, 1H), 7.30 (d, 2H), 7.38 (t, 1H), 7.58 (d, 1H), 7.70 (s, 1H), 7.79 (t, 1H), 8.71 (d, 1H)

Mass Spectra (m/e): 496 (M+H).

TABLE 7

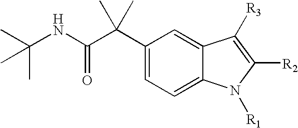

| | | | | Mass |
|---|---|---|---|---|
| | | | | Spectral |
| | | | | Data m/e |
| Example | R₁ | R₂ | R₃ | (M + H) |
| 141 | 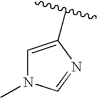 | 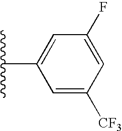 | H | 501 |
| 142 | 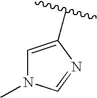 | 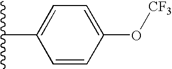 | H | 499 |
| 143 | 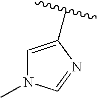 | 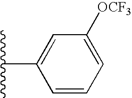 | H | 499 |
| 144 | 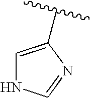 | 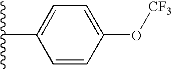 | H | 485 |
| 145 | 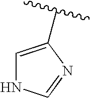 | 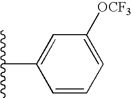 | H | 485 |
| 146 | 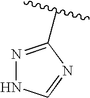 | 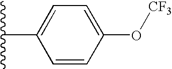 | H | 486 |
| 147 | 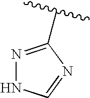 | 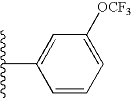 | H | 486 |
| 148 | 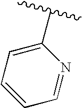 | 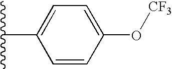 | H | 496 |
| 149 | 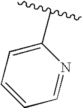 | 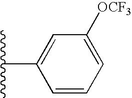 | H | 496 |
| 150 | 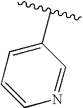 | 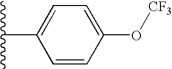 | H | 496 |
| 151 | 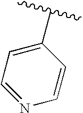 | 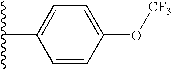 | H | 496 |
| 152 | 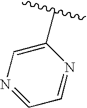 | 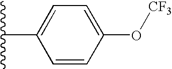 | H | 497 |

TABLE 8

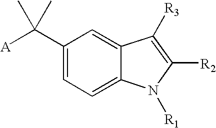

| | | | | | Mass |
|---|---|---|---|---|---|
| | | | | | Spectral |
| | | | | | Data m/e |
| Example | A | R₁ | R₂ | R₃ | (M + H) |
| 153 | —CON(CH₃)₂ | —CH₂CONHt-Bu | 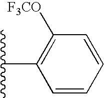 | H | 504 |
| 154 | 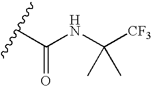 | —CH₂CONHt-Bu | 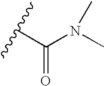 | H | 497 |
| 155 | 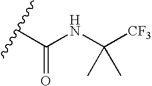 | —CH₂CONHt-Bu | 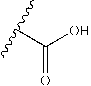 | H | 470 |
| 156 | 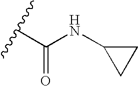 | —CH₂CONHt-Bu | 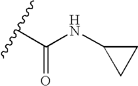 | H | 439 |
| 157 | 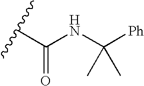 | —CH₂CONHt-Bu | 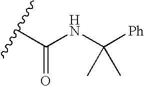 | H | 595 |
| 158 | 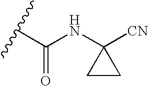 | —CH₂CONHt-Bu | 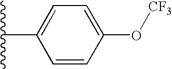 | H | 541 |
| 159 | 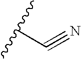 | —CH₂CONHt-Bu | 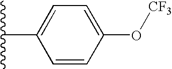 | H | 458 |
| 160 | 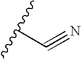 | —CH₂CONHt-Bu | 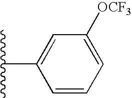 | H | 458 |
| 161 | 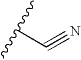 | —CH₂CONHt-Bu | -tBu | H | 354 |
| 162 | 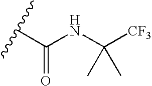 | —CH₂CONHt-Bu | 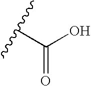 | H | 470 |
| 163 | 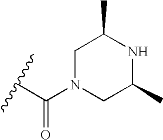 | —CH₂CONHt-Bu | 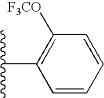 | H | 573 |

What is claimed is:

1. The compounds of this invention are represented by Formula I: 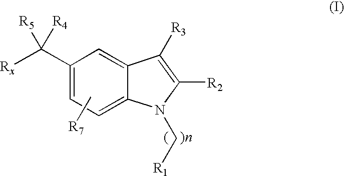
and pharmaceutically acceptable salts, individual enantiomers and diastereomers thereof wherein:
Rₓ is 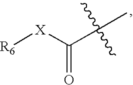
CN, or CH₂OH;
n is 0-3, where when n=0, R₁ is not H;
X═NR₆, O or is a bond;
R₁ is selected from:
a) hydrogen, C₁-C₆-alkyl or C₃-C₇-cycloalkyl, both optionally substituted with 1 to 3 groups of a substituent selected from C₁-C₄-perfluoroalkyl, C₁-C₆-alkyl, F, Cl, Br, NH₂, NHR₈, NR₈R₉, OH, OR₈, CONHR₈, COOR₈, COR₈, SR₈, SO₂R₁₀, SO₂NHR₈, C₆-C₁₀ aryl or C₅-C₁₀ heteroaryl,
b) C₆-C₁₀ aryl or C₅-C₁₀ heterocycle, both optionally substituted with 1 to 3 groups of a substituent selected from C₁-C₄-perfluoroalkyl, C₁-C₆-alkyl, C₃-C₇-cycloalkyl, F, Cl, Br, NH₂, NHR₈, NR₈R₉, OH, OR₈, CONHR₈, CONR₈R₉, COOR₈, and COR₈,
c) CONR₈R₉, COOR₈ or COR₈, and
d) SOR₁₀, SO₂R₁₀, or SO₂NHR₈;
R₂ is selected from:
(b) C₁-C₆-alkyl, C₃-C₇-cycloalkyl, C₆-C₁₀ aryl or (CH₂)ₙC₅-C₁₀ heterocycle, said alkyl, cycloalkyl, aryl and heteroaryl optionally substituted with 1 to 3 groups of a substituent selected from (O)₀₋₁C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, and COR₈;
(b) CONR₈R₉, COOR₈ or COR₈
R₃ is selected from:
(a) H, C₁-C₄-perfluoroalkyl, C₁-C₆-alkyl and C₃-C₇-cycloalkyl, said alkyl and cycloalkyl optionally substituted with 1 to 3 groups of a substituent selected from C₁-C₄-perfluoroalkyl, C₁-C₆-alkyl, F, Cl, Br, NH₂, NHR₈, NR₈R₉, OR₈, CONHR₈, COOR₈, COR₈, SR₈, SO₂R₁₀, SO₂NHR₈, C₆-C₁₀ aryl and C₅-C₁₀ heteroaryl, NHC(O)(CH₂)ₙOR₈;
(b) CN, CONHR₈, CONR₈R₉, COOR₈ or COR₈;
(c) SOR₁₀, SO₂R₁₀, SR₈, or SO₂ NR₈R₉;
(d) C₆-C₁₀ aryl or (CH₂)ₙC₅-C₁₀ heterocyclyl, both optionally substituted with 1 to 3 groups of C₁-C₄-perfluoroalkyl, C₁-C₆-alkyl, C₆-C₁₀ aryl, F, Cl, Br, CN, NH₂, NHR₈, NR₈R₉, OH, OR₈, SO₂R₆, CONHR₈, CONR₈R₉, COOR₈, or COR₈;
R₄ and R₅ are each independently selected from H and C₁-C₆-alkyl, said alkyl optionally substituted with 1 to 3 groups of a substituent selected from C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, and COR₈, or R₄ and R₅ join to form a 3-7 member carbocyclic or heterocyclic ring;
R₆ is selected from H, C₁-C₆-alkyl, C₃-C₇-cycloalkyl, C₁-C₄-alkylaryl, and (CH₂)ₙC₅-C₁₀ heterocyclyl, said alkyl, cycloalkyl, alkylaryl, aryl and heteroaryl optionally substituted with 1 to 3 groups of a substituent selected from C₁-C₄-perfluoroalkyl, CN, F, Cl, Br, NH₂, C₆-C₁₀ aryl, NHR₇, NR₈R₉, OH, OR₈, CONHR₈, CONR₈R₉, COOR₈ and COR₈;
R₇ is selected from H, C₁-C₄-alkyl, C₃-C₇-cycloalkyl, C₁-C₄-perfluoroalkyl, F, Cl, Br, I, NR₈R₉, OR₈, CONHR₈, CONR₈R₉, COOR₈, and COR₈;
R₈ and R₉ are each independently selected from H, C₁-C₆-alkyl, C₃-C₇-cycloalkyl, N(R₆)₂, SO₂R₆, —COOR₆, —C(O)C(R₆)₂OCO₂R₆, C(O)C(C₃₋₇ cycloalky)OR₆, C(O)C(C₃₋₇cycloalky)OCO₂R₆, (CH₂)ₙC₆-C₁₀ aryl and (CH₂)ₙC₅-C₁₀ heterocycle, said alkyl, cycloalkyl, aryl and hereroaryl optionally substituted with 1 to 3 groups selected from (O)₀₋₁C₁-C₄-perfluoroalkyl, C₁-C₆-alkyl, F, Cl, Br, CN, NH₂, NHR₈, NR₈R₉, OH, OR₈, (CH₂)ₙC₆-C₁₀ aryl, CONHR₈, CONR₈R₉, COOR₈, or COR₈; and
R₁₀ is selected from C₁-C₄-alkyl, C₃-C₇-cycloalkyl, C₆-C₁₀ aryl and C₅-C₁₀ heteroaryl, said alkyl, cycloalkyl, aryl and heteroaryl optionally substituted with 1 to 3 groups selected from (O)₀₋₁C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, or COR₈.

2. The compound according to claim 1 wherein R_{X} is

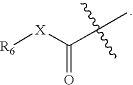

3. The compound according to claim 2 wherein X is NR₆.

4. The compound according to claim 2 wherein X is —O—.

5. The compound according to claim 2 wherein X is a bond.

6. The compound according to claim 1 wherein R₆ is hydrogen, C₁-C₆-alkyl, C₃-C₆-cycloalkyl, or (CH₂)ₙC₅-C₁₀ heterocyclyl, n is 0 or 1, R₁ is C(O)OR₈, C₁-C₆-alkyl, C(O)N(R₈)₂, C₅₋₁₀ heterocycle, or —SO₂R₁₀, and R₂ is C₁-C₆-alkyl, C₆-C₁₀ aryl, or (CH₂)ₙC₅-C₁₀ heterocycle.

7. The compound according to claim 2 wherein R₆ is hydrogen, C₁-C₆-alkyl, C₃-C₆-cycloalkyl, or (CH₂)ₙC₅-C₁₀ heterocyclyl, n is 0 or 1, R₁ is C(O)OR₈, C(O)R₈, C₁-C₆-alkyl, C(O)N(R₈)₂, C₅₋₁₀ heterocycle, or —SO₂R₁₀, and R₂ is C₁-C₆-alkyl, C₆-C₁₀ aryl, or (CH₂)ₙC₅-C₁₀ heterocycle.

8. The compound according to claim 6 wherein R₆ is hydrogen, or C₁-C₆-alkyl, n is 0 or 1, R₁ is C(O)OR₈, C(O)R₈, C₁-C₆-alkyl, or C(O)N(R₈)₂, and R₂ is C₁-C₆-alkyl, or C₆-C₁₀ aryl.

9. The compound according to claim 7 wherein R₆ is hydrogen, or C₁-C₆-alkyl, n is 0 or 1, R₁ is C(O)OR₈, C(O)R₈, C₁-C₆-alkyl, or C(O)N(R₈)₂, and R₂ is C₁-C₆-alkyl, or C₆-C₁₀ aryl.

10. The compound according to claim 1 wherein R₃ is H, C₁-C₆-alkyl, CN, CONR₈R₉, SO₂R₁₀, —COOR₈, —COR₈, or (CH₂)ₙC₅-C₁₀ heterocycle,

11. The compound according to claim 10 wherein R₃ is H, or C₁-C₆-alkyl.

12. The compound of structural formula II according to claim 1: 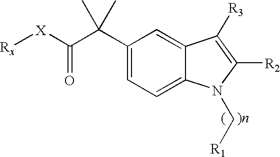
and pharmaceutically acceptable salts, individual enantiomers and diastereomers thereof wherein:
R₂ is C₁-C₆-alkyl, C₆-C₁₀ aryl or (CH₂)ₙC₅-C₁₀ heterocycle, said alkyl, aryl, and heteroaryl optionally substituted with 1 to 3 groups of a substituent selected from (O)₀₋₁C₁-C₄-perfluoroalkyl, C₁-C₆-alkyl, F, Cl, Br, CN, NH₂, NHR₈, NR₈R₉, OH, OR₈, CONHR₈, CONR₈R₉, COOR₈, and COR₈; and X, Rₓ, R₁, R₂ and R₃ are as described in claim 1.

13. The compound according to claim 12 wherein R₂ is C₆-C₁₀ aryl; R₁ is C₁-C₆-alkyl, C(O)N(R₈)₂, C₅₋₁₀ heterocycle, COOR₈ or COR₈, R₃ is H, or C₁-C₆-alkyl, and R₆ is hydrogen, C₁-C₆-alkyl, C₃-C₆-cycloalkyl, or (CH₂)ₙC₅-C₁₀ heterocyclyl.

14. The compound according to claim 13 wherein R₂ is phenyl.

15. A compound selected from Tables A, B, C, D and E:
TABLE A 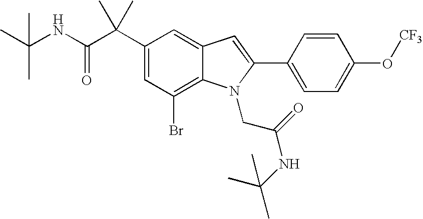 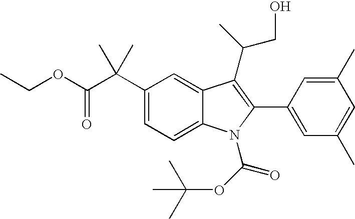 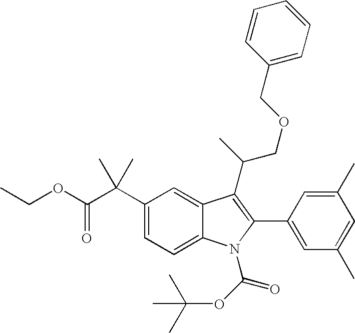 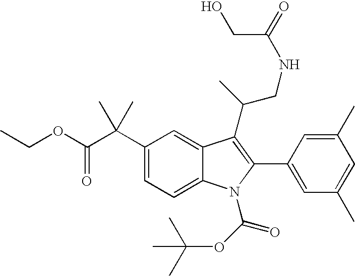 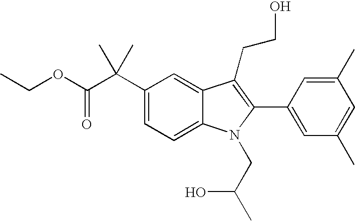 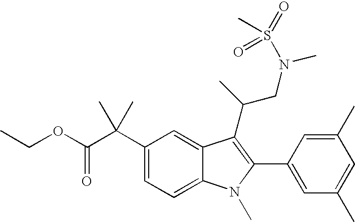 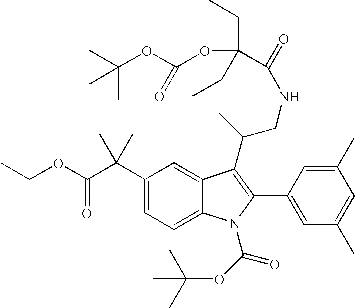 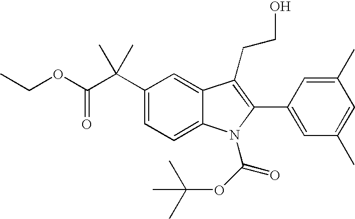 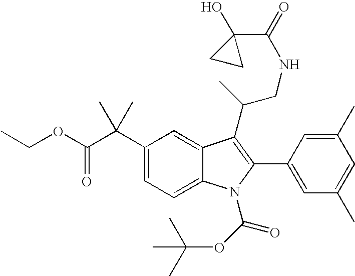 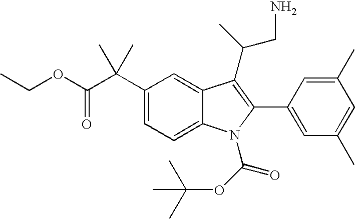 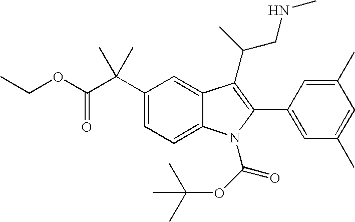 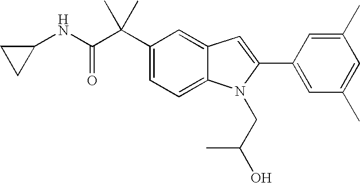 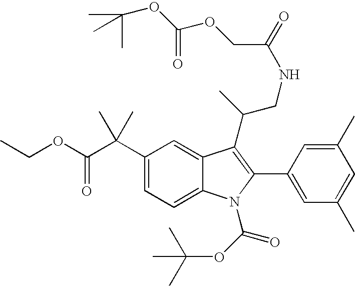 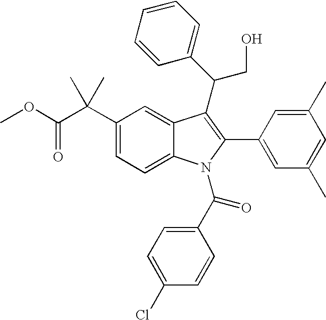 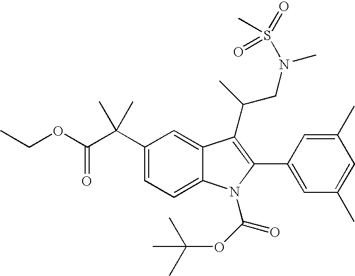 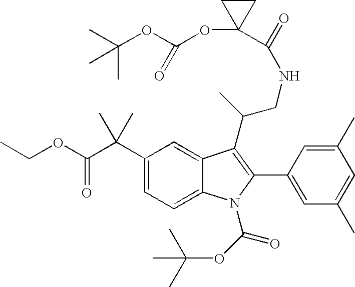 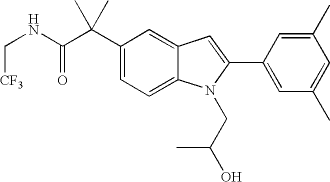 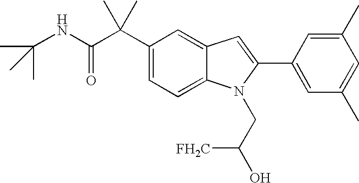 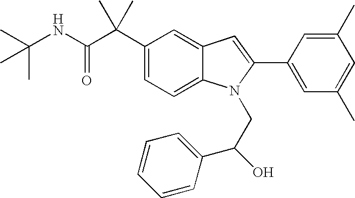 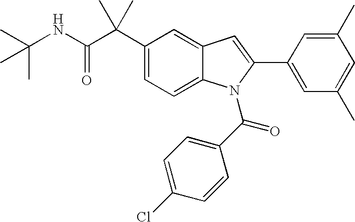 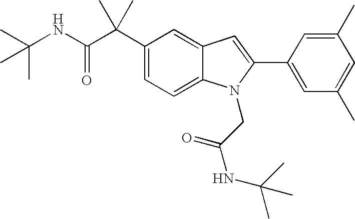 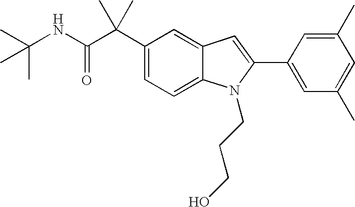 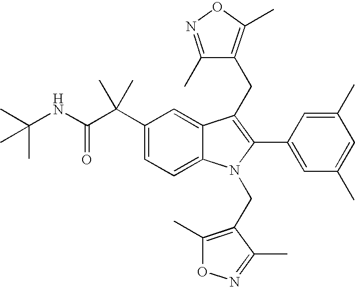 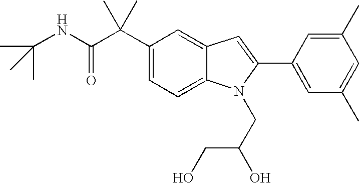 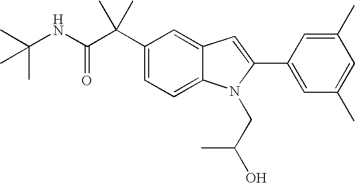 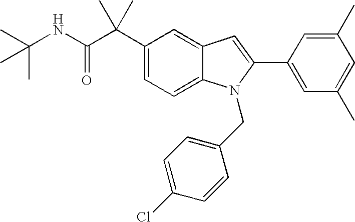 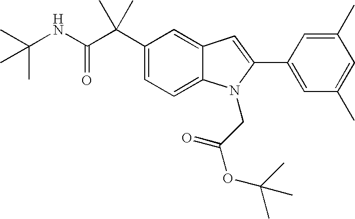 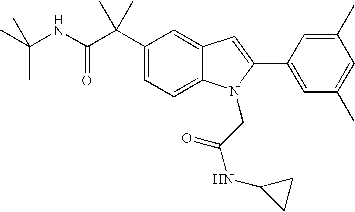 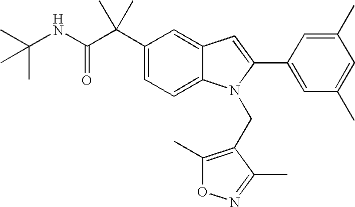 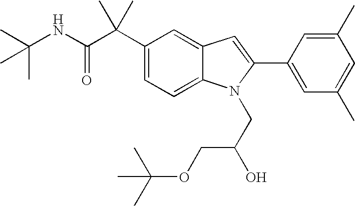 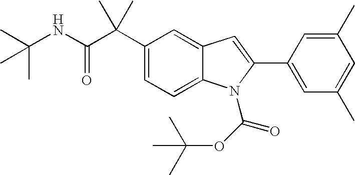 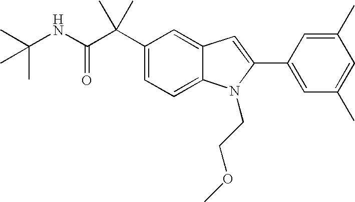 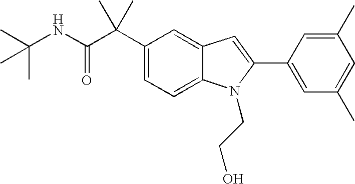 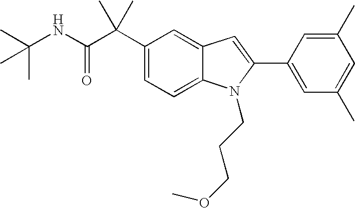 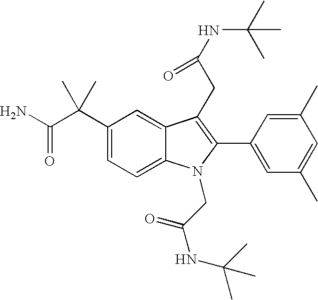 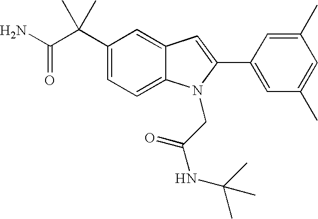 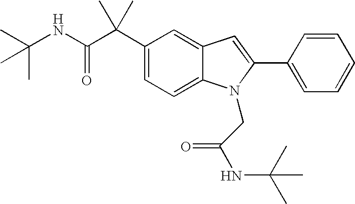 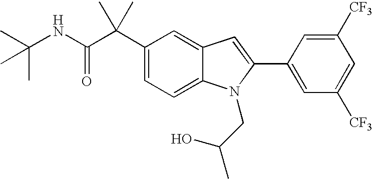 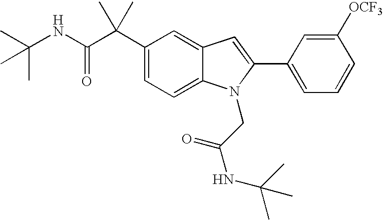 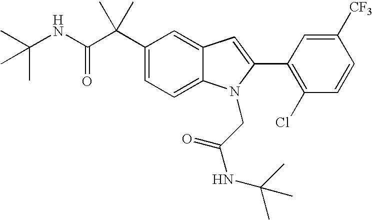 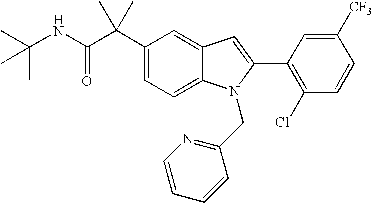 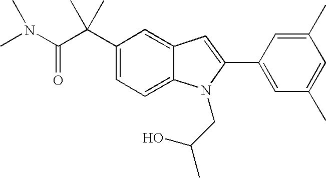 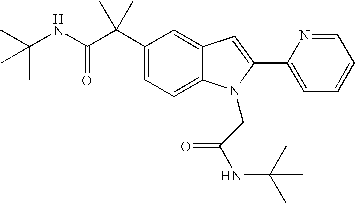 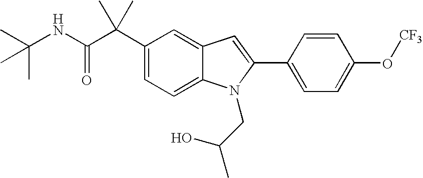 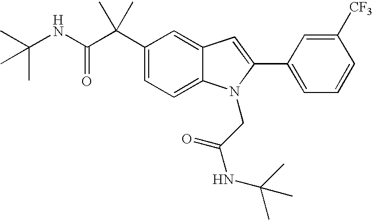 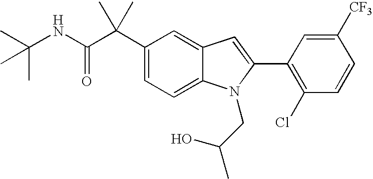 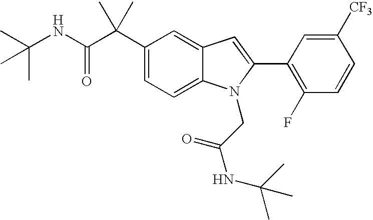 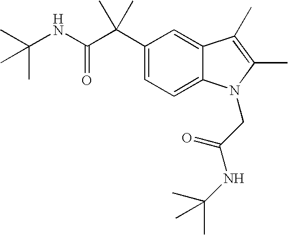 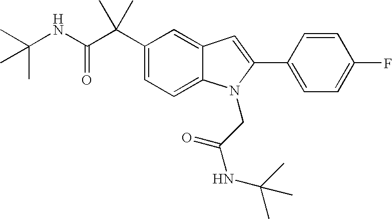 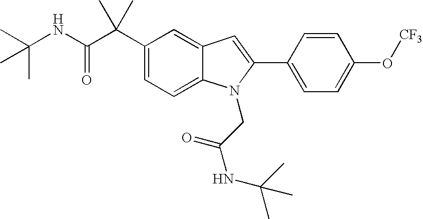 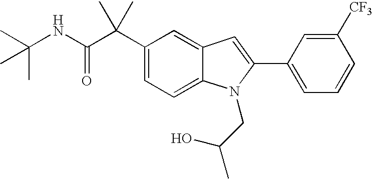 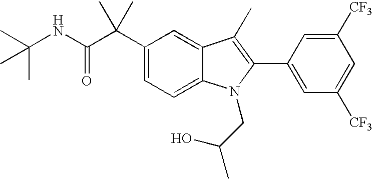 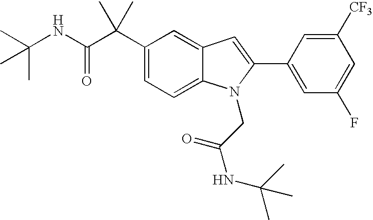 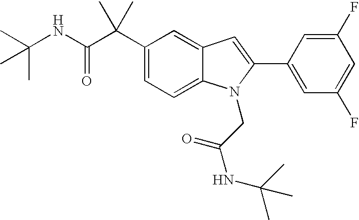 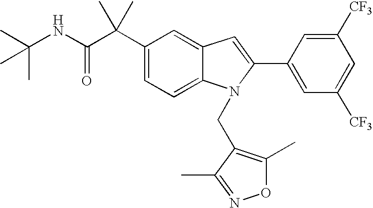 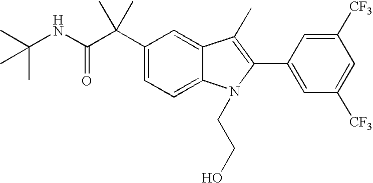 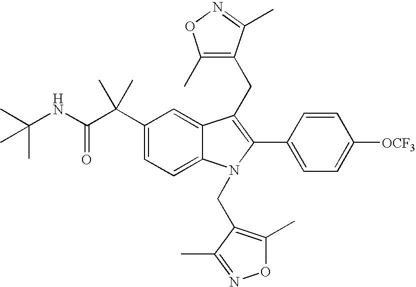 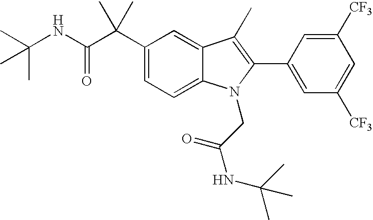 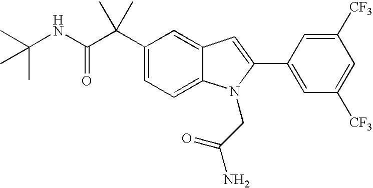 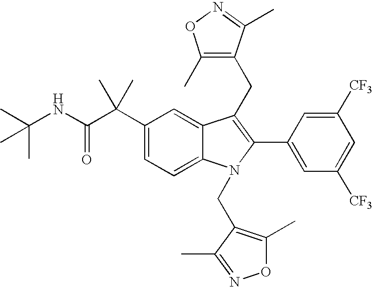 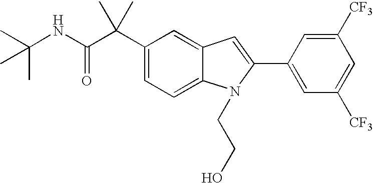 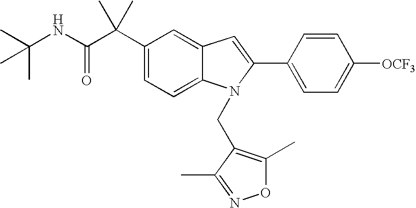 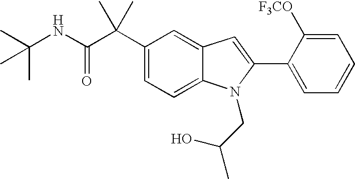 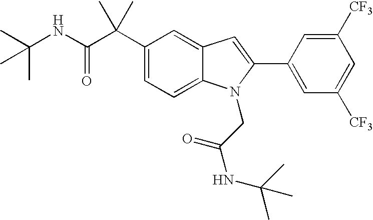 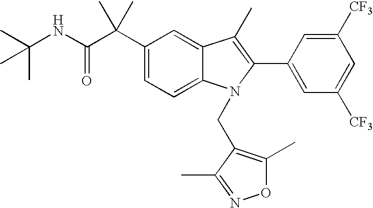 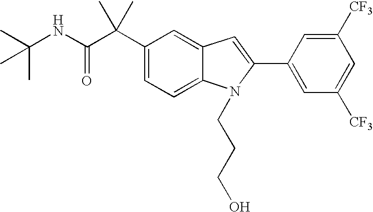 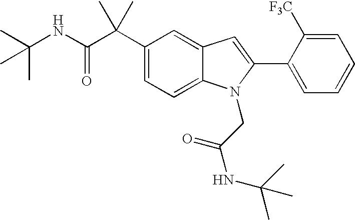 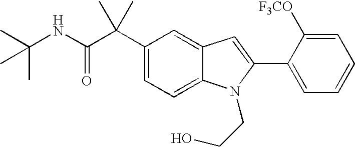 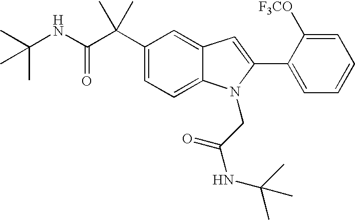 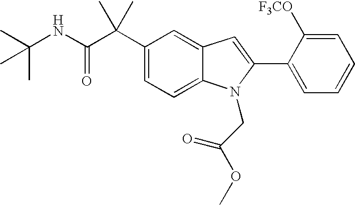 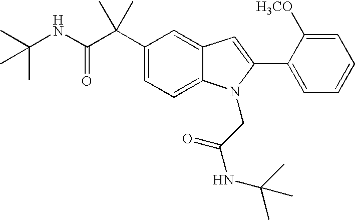 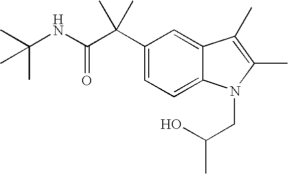 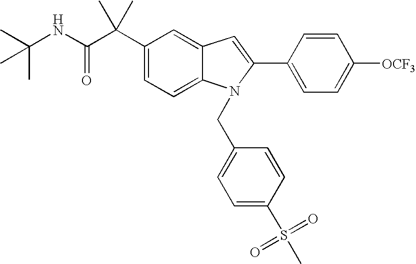 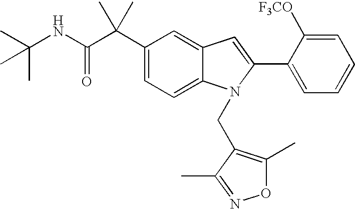 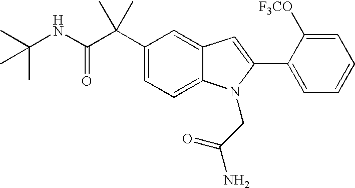 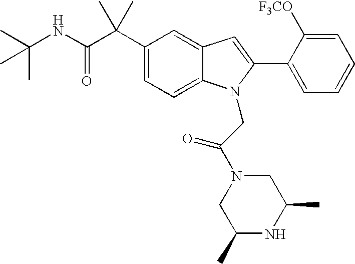 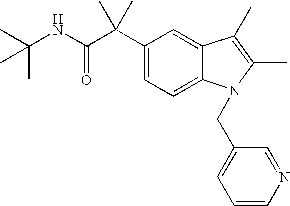 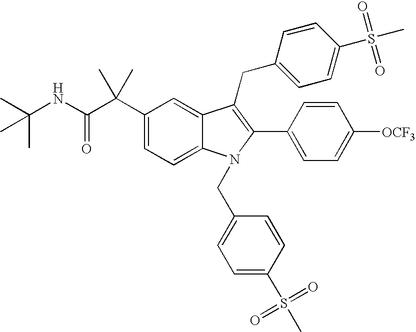 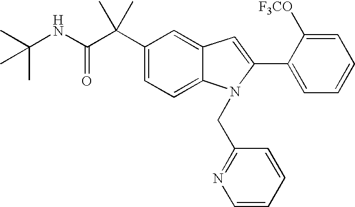 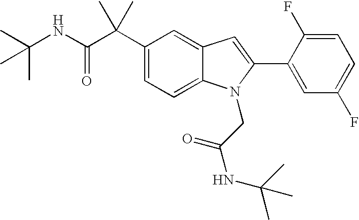 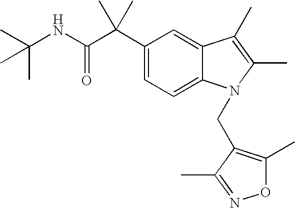 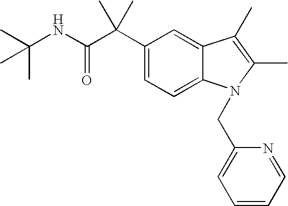 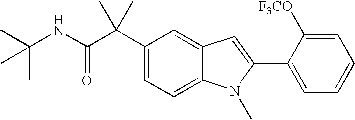 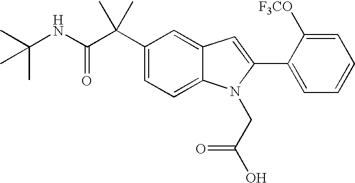 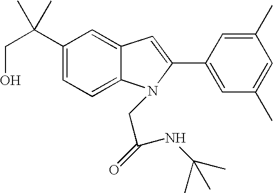 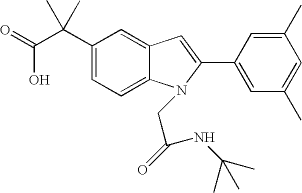 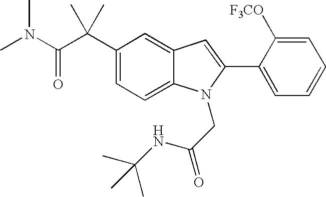 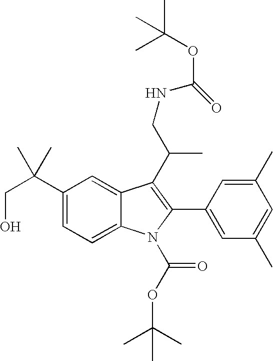 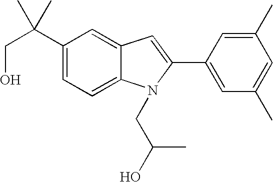 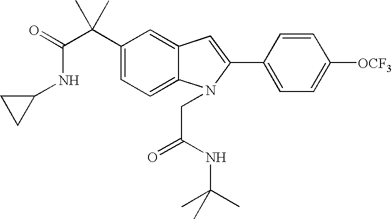 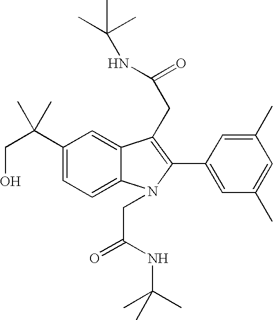 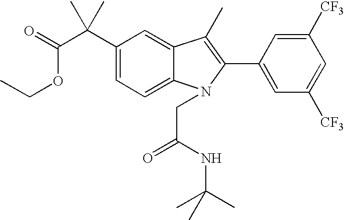 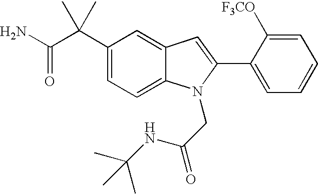 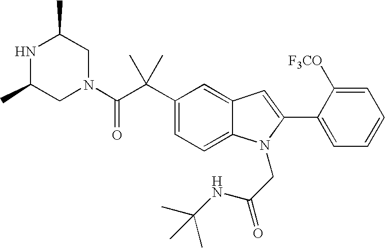
TABLE B 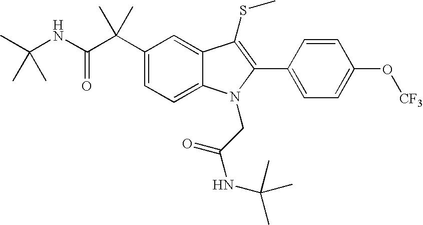 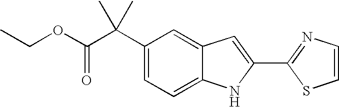 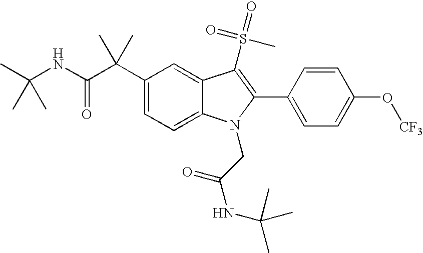 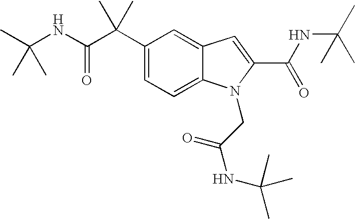 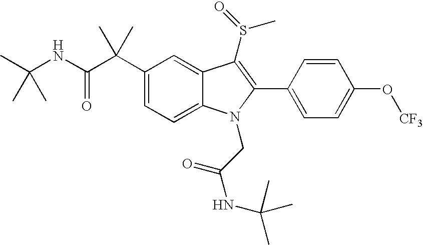 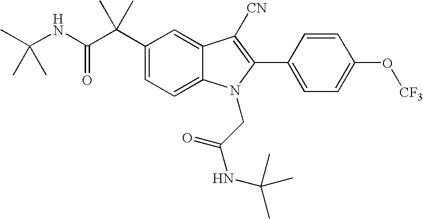
TABLE C

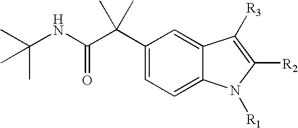

| Example | R₁ | R₂ | R₃ |
|---|---|---|---|
| 121 | —CH₂CONH-t-Bu | 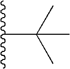 | H |
| 122 | 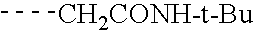 | 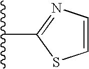 | H |
| 123 | —CH₂CONH-t-Bu | 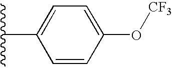 | CO₂H |
| 124 | —CH₂CONH-t-Bu | 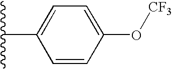 | CONH₂ |
| 125 | —CH₂CONH-t-Bu | 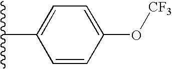 | SPh |
| 126 | —CH₂CONH-t-Bu | 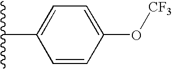 | S(O)Ph |
| 127 | —CH₂CONH-t-Bu | 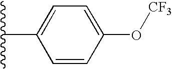 | SO₂Ph |
| 128 | —CH₂CONH-t-Bu | 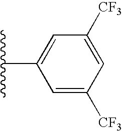 | SO₂Me |
| 129 | —CH₂CONH-t-Bu | 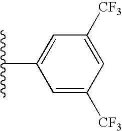 | SO₂Ph |
| 130 | 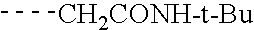 | 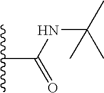 | CN |
| 131 | 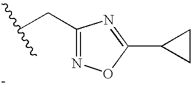 | 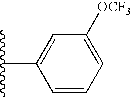 | H |
| 132 | —CH₂CONH-t-Bu | 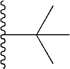 | H |
| 133 | 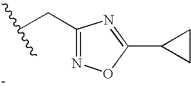 | 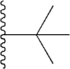 | H |
| 134 | 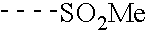 | 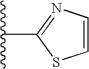 | H |
| 135 | —SO₂Me | 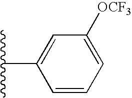 | —COMe |
| 136 | —CH₂CONH-t-Bu | 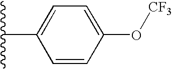 | 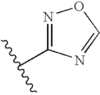 |
| 137 | —CH₂CONH-t-Bu | 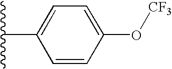 | 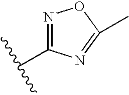 |
| 138 | —CH₂CONH-t-Bu | 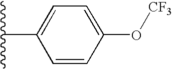 | 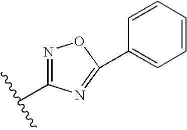 |
| 139 | —CH₂COOH | 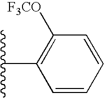 | H |

TABLE D

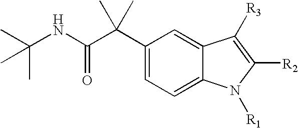

| Example | R₁ | R₂ | R₃ |
|---|---|---|---|
| 140 | 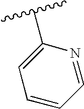 | 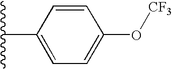 | H |
| 141 | 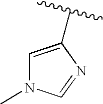 | 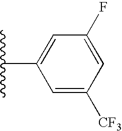 | H |
| 142 | 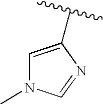 | 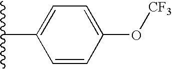 | H |
| 143 | 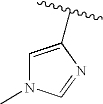 | 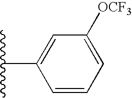 | H |
| 144 | 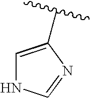 | 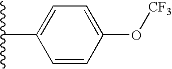 | H |
| 145 | 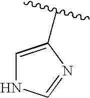 | 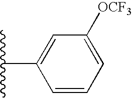 | H |
| 146 | 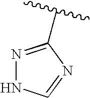 | 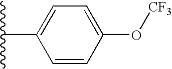 | H |
| 147 | 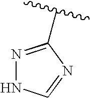 | 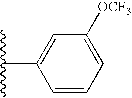 | H |
| 148 | 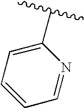 | 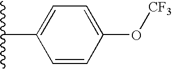 | H |
| 149 | 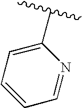 | 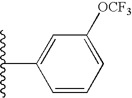 | H |
| 150 | 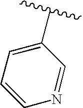 | 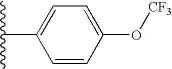 | H |
| 151 | 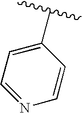 | 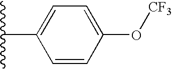 | H |
| 152 | 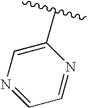 | 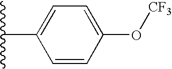 | H |

TABLE E

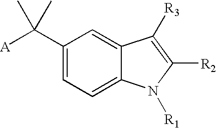

| Example | A | R₁ | R₂ | R₃ |
|---|---|---|---|---|
| 153 | —CON(CH₃)₂ | —CH₂CONHt-Bu | 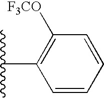 | H |
| 154 | 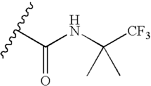 | —CH₂CONHt-Bu | 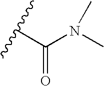 | H |
| 155 | 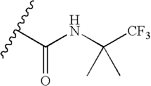 | —CH₂CONHt-Bu | 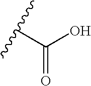 | H |
| 156 | 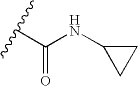 | —CH₂CONHt-Bu | 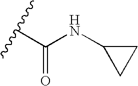 | H |
| 157 | 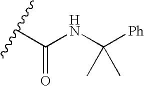 | —CH₂CONHt-Bu | 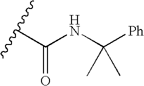 | H |
| 158 | 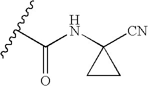 | —CH₂CONHt-Bu | 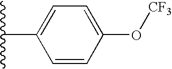 | H |
| 159 | 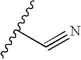 | —CH₂CONHt-Bu | 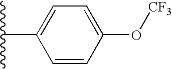 | H |
| 160 | 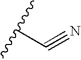 | —CH₂CONHt-Bu | 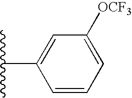 | H |
| 161 | 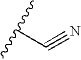 | —CH₂CONHt-Bu | -tBu | H |
| 162 | 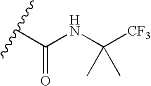 | —CH₂CONHt-Bu | 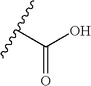 | H |
| 163 | 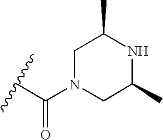 | —CH₂CONHt-Bu | 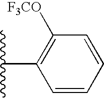 | H |

and pharmaceutically acceptable salts thereof and individual enantiomers and diastereomers thereof.

16. The compound according to claim 15 which is:

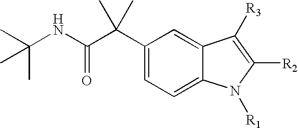

| R₁ | R₂ | R₃ |
|---|---|---|
| —CH₂CH(OH)CH₃ | 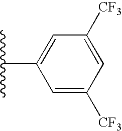 | H |
| —CH₂CH(OH)CH₃ | 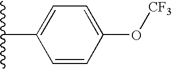 | H |
| —CH₂CONH-t-Bu | 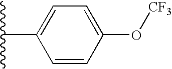 | H |
| —CH₂CONHtBu | 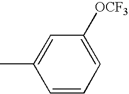 | H |
| —CH₂CH(OH)CH₃ | 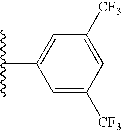 | CH₃ |
| 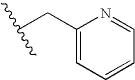 | 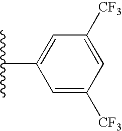 | H |
| —CH₂CONHtBu | 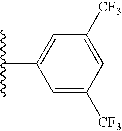 | H |
| —CH₂CH₂OH | 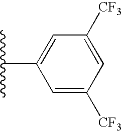 | H |
| —CH₂CONH-t-Bu | 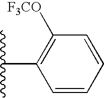 | H |
| 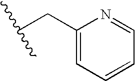 | 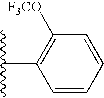 | H |
| 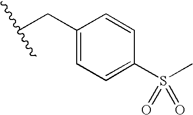 | 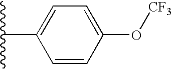 | H |
| CH₃ | 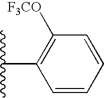 | H |

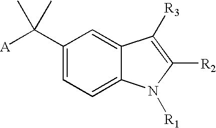
| A | R₁ | R₂ | R₃ |
|---|---|---|---|
| —CONH-c-Pr | —CH₂CONHt-Bu | 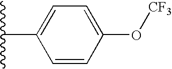 | H |

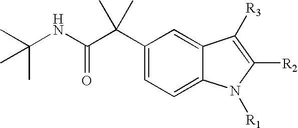
| R₁ | R₂ | R₃ |
|---|---|---|
| —CH₂CONH-t-Bu | 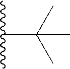 | H |
| 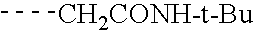 | 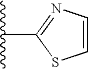 | H |
| —CH₂CONH-t-Bu | 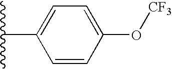 | CO₂H |
| —CH₂CONH-t-Bu | 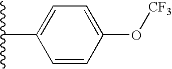 | CONH₂ |
| —CH₂CONH-t-Bu | 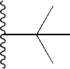 | H |

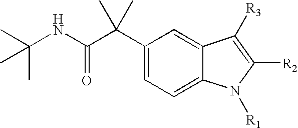
| R₁ | R₂ | R₃ |
|---|---|---|
| 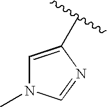 | 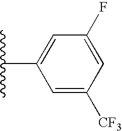 | H |
| 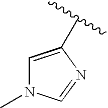 | 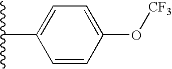 | H |
| 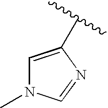 | 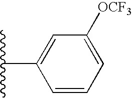 | H |
| 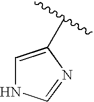 | 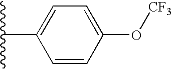 | H |
| 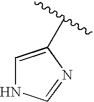 | 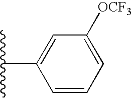 | H |

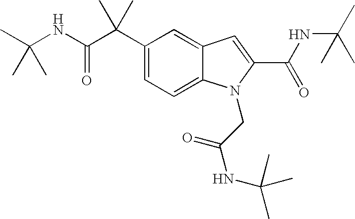

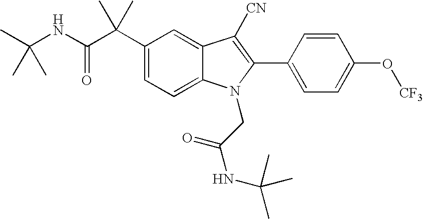

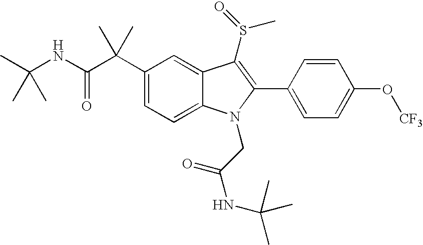

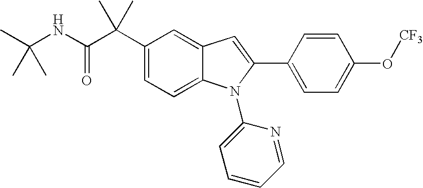

and pharmaceutically acceptable salts, individual enantiomers and diastereomers thereof.

17. A pharmaceutical composition comprising an inert carrier and an effective amount of a compound according to claim 1.

18. A method for treating or preventing chronic or neuropathic pain in a mammalian patient in need thereof comprising administering to said patient a therapeutically effective amount, or a prophylactically effective amount, of a compound according to claim 1, or a pharmaceutically acceptable salt thereof.

19. A method for treating or controlling epilepsy in a mammalian patient in need thereof which comprises administering to the patient a therapeutically effective amount of the compound of claim 1, or a pharmaceutically acceptable salt thereof.

20. A method for enhancing the quality of sleep in a mammalian patient in need thereof which comprises administering to the patient a therapeutically effective amount of the compound of claim 1, or a pharmaceutically acceptable salt thereof.
